SEQUENCE LISTING (56 sequences)

SEQ ID NO: 1 - DNA, 20 bp, synthetic construct
  source 1..20: mol_type = other DNA
acccttctccggccgctgac

SEQ ID NO: 2 - DNA, 90 bp, synthetic construct
  source 1..90: mol_type = other DNA
tccgctctggggcgcgcacaccctcgcccgcacttctcccctcgtccggcacggactgaccgcacacgaataacacatccgaccagagct

SEQ ID NO: 3 - DNA, 20 bp, synthetic construct
  source 1..20: mol_type = other DNA
agacggggtttcggcatgtt

SEQ ID NO: 4 - DNA, 20 bp, synthetic construct
  source 1..20: mol_type = other DNA
attgtgtaggctggtctcga

SEQ ID NO: 5 - DNA, 20 bp, synthetic construct
  source 1..20: mol_type = other DNA
ggggcaggaaggaaggcaga

SEQ ID NO: 6 - DNA, 20 bp, synthetic construct
  source 1..20: mol_type = other DNA
gtggtaaacagagcgcctag

SEQ ID NO: 7 - DNA, 15 bp, Homo sapiens
  source 1..15: mol_type = other DNA
  variation 8: note = y is 'c or t/u'
  misc_difference 9: note = n is a variable number of any nucleotide
gtgaacgynctacac

SEQ ID NO: 8 - DNA, 12 bp, Mus musculus
  source 1..12: mol_type = genomic DNA
  variation 6: note = r is 'a or g'
ttcctrgaaagc

SEQ ID NO: 9 - DNA, 12 bp, Homo sapiens
  source 1..12: mol_type = genomic DNA
  variation 7: note = k is 'g or t/u'
gctttckaggaa

SEQ ID NO: 10 - DNA, 10 bp, Mus musculus
  source 1..10: mol_type = genomic DNA
  variation 6..7: note = r is 'a or g'
  variation 9: note = v is 'a or c or g'
  variation 10: note = d is 'a or g or t/u'
ttcctrravd

SEQ ID NO: 11 - DNA, 12 bp, Homo sapiens
  source 1..12: mol_type = genomic DNA
  variation 7: note = k is 'g or t/u'
gctttckaggaa

SEQ ID NO: 12 - DNA, 10 bp, Homo sapiens
  source 1..10: mol_type = genomic DNA
gaccacccac

SEQ ID NO: 13 - DNA, 16 bp, Homo sapiens
  source 1..16: mol_type = genomic DNA
  variation 1: note = k is 'g or t/u'
  variation 11: note = r is 'a or g'
  variation 13: note = r is 'a or g'
kaccccccacrcraag

SEQ ID NO: 14 - DNA, 10 bp, Homo sapiens
  source 1..10: mol_type = genomic DNA
ggcacgtgcc

SEQ ID NO: 15 - DNA, 15 bp, Homo sapiens
  source 1..15: mol_type = genomic DNA
awcgaaaccgaaact

SEQ ID NO: 16 - DNA, 11 bp, Mus musculus
  source 1..11: mol_type = genomic DNA
aattaattaat

SEQ ID NO: 17 - DNA, 10 bp, Homo sapiens
  source 1..10: mol_type = genomic DNA
ctatttatag

SEQ ID NO: 18 - DNA, 10 bp, Homo sapiens
  source 1..10: mol_type = genomic DNA
ctaaaaatag

SEQ ID NO: 19 - DNA, 10 bp, Homo sapiens
  source 1..10: mol_type = genomic DNA
ctaaaaatag

SEQ ID NO: 20 - DNA, 14 bp, Mus musculus
  source 1..14: mol_type = genomic DNA
  misc_difference 2: note = n is a variable number of any nucleotide
  misc_difference 12: note = n is a variable number of any nucleotide
  variation 9: note = v is 'a or c or g'
  variation 11: note = m is 'a or c'
anccacttvamntt

SEQ ID NO: 21 - DNA, 14 bp, Homo sapiens
  source 1..14: mol_type = genomic DNA
  misc_difference 12: note = n is a variable number of any nucleotide
  variation 3: note = y is 'c or t/u'
  variation 8: note = w is 'a or t/u'
  variation 10: note = s is 'c or g'
ttyacgcwtsantt

SEQ ID NO: 22 - DNA, 13 bp, Homo sapiens
  source 1..13: mol_type = genomic DNA
  variation 1: note = r is 'a or g'
rggtcaaaggtca

SEQ ID NO: 23 - DNA, 12 bp, Homo sapiens
  source 1..12: mol_type = genomic DNA
  misc_difference 6: note = n is a variable number of any nucleotide
  variation 7: note = r is 'a or g'
ggtcanrggtca

SEQ ID NO: 24 - DNA, 14 bp, Mus musculus
  source 1..14: mol_type = genomic DNA
  misc_difference 7: note = n is a variable number of any nucleotide
  variation 6: note = d is 'a or g or t/u'
acaatdncattgtt

SEQ ID NO: 25 - DNA, 16 bp, Homo sapiens
  source 1..16: mol_type = genomic DNA
  variation 7: note = r is 'a or g'
  variation 9: note = k is 'g or t/u'
aacaatrtkcagtgtt

SEQ ID NO: 26 - DNA, 12 bp, Homo sapiens
  source 1..12: mol_type = genomic DNA
  misc_difference 6: note = n is a variable number of any nucleotide
ggaaangaaact

SEQ ID NO: 27 - DNA, 11 bp, Homo sapiens
  source 1..11: mol_type = genomic DNA
  misc_difference 6: note = n is a variable number of any nucleotide
tggcangccaa

SEQ ID NO: 28 - DNA, 11 bp, Homo sapiens
  source 1..11: mol_type = genomic DNA
  misc_difference 6: note = n is a variable number of any nucleotide
aattgncaatt

SEQ ID NO: 29 - DNA, 10 bp, Homo sapiens
  source 1..10: mol_type = genomic DNA
cgaccaccga

SEQ ID NO: 30 - DNA, 12 bp, Mus musculus
  source 1..12: mol_type = genomic DNA
cccccggggggg

SEQ ID NO: 31 - DNA, 15 bp, Homo sapiens
  source 1..15: mol_type = genomic DNA
  variation 1: note = v is 'a or c or g'
  variation 12: note = k is 'g or t/u'
  variation 14: note = y is 'c or t/u'
vctccccccgckgyg

SEQ ID NO: 32 - DNA, 13 bp, Mus musculus
  source 1..13: mol_type = genomic DNA
  variation 1: note = m is 'a or c'
mcccccggggggg

SEQ ID NO: 33 - DNA, 1240 bp, Homo sapiens
  source 1..1240: mol_type = genomic DNA
gattacgaggtcaggagttcaagaccagcctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatactctgtcgcaaaaaaaaaaaagaatactggaggctgggcgaggtggctcacacctgtaatcccagcattttgggatgccagaggcgggcggaatatcttgagctcaggagttcgagaccagcctacacaatatgctccaaacgccgcctctacaaaacatacagaaactagccgggtgtggtggcgtgcccctgtggtcctagctacttgggaggttgaggcgggaggatcgcttgagctcgggaggtcgaggctgcaatgagccgagatggtgccactgcactctgacgacagagcgagactccgtctcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattcc

SEQ ID NO: 34 - DNA, 20 bp, synthetic construct
  source 1..20: mol_type = other DNA
aacatgccgaaaccccgtct

SEQ ID NO: 35 - DNA, 20 bp, synthetic construct
  source 1..20: mol_type = other DNA
tcgagaccagcctacacaat

SEQ ID NO: 36 - DNA, 20 bp, synthetic construct
  source 1..20: mol_type = other DNA
tctgccttccttcctgcccc

SEQ ID NO: 37 - DNA, 40 bp, synthetic construct
  source 1..40: mol_type = other DNA
acccttctccggccgctgacagacggggtttcggcatgtt

SEQ ID NO: 38 - DNA, 39 bp, synthetic construct
  source 1..39: mol_type = other DNA
acccttctccggccgctgacattgtgaggctggtctcga

SEQ ID NO: 39 - DNA, 40 bp, synthetic construct
  source 1..40: mol_type = other DNA
acccttctccggccgctgacggggcaggaaggaaggcaga

SEQ ID NO: 40 - DNA, 110 bp, synthetic construct
  source 1..110: mol_type = other DNA
tccgctctggggcgcgcacaccctcgcccgcacttctcccctcgtccggcacggactgaccgcacacgaataacacatccgaccagagctagacggggtttcggcatgtt

SEQ ID NO: 41 - DNA, 110 bp, synthetic construct
  source 1..110: mol_type = other DNA
tccgctctggggcgcgcacaccctcgcccgcacttctcccctcgtccggcacggactgaccgcacacgaataacacatccgaccagagctattgtgtaggctggtctcga

SEQ ID NO: 42 - DNA, 110 bp, synthetic construct
  source 1..110: mol_type = other DNA
tccgctctggggcgcgcacaccctcgcccgcacttctcccctcgtccggcacggactgaccgcacacgaataacacatccgaccagagctggggcaggaaggaaggcaga

SEQ ID NO: 43 - DNA, 1960 bp, Homo sapiens
  source 1..1960: mol_type = genomic DNA
  variation 4..23: note = Binding sequence 1
  variation 30..75: note = Enhancer
  variation 76..80: note = Promoter
  variation 1716..1775: note = Exon 1 TCFA-201
tctctaggcgctctgtttaccactctatggtcgcctgctcccgccccgccccttcccagtcattgtctggaggagcagccgcggccgcagctccttctctttataagcccgcagttcccggatgtgaatggattacaatgtatctttcagggaaacctattattatcaatgtgactccacgggggagtccatggtgatgatgatgaggaggaggatgatgatgatgagacacctctaaacttggaacaagtttaagactttatgagagaagaaaaaaaatcaccaacaagaattgtttgaggaaaaattataactatcctgtgttcatttttttttttataaacaataagaaaaagttgttggatttttttttaatgatttcttttttgggggagggaattttgttgcagttttatggtggaaaatgcaaaaaccagagccaggtgcataatcttgtaatctgtggatatccctggagcaggactgagtaccagttaaaatactttttggggatacacatgtgagatactaagtacttgcagaagatttttgtctctctttttaaagtctctttccttggaatattgtgagaatatttgtggccatttaaggtaacgtttcaatttgccgtcagagtaaactgtttgtaattgaatttaatttttaaaatgtcgatcccgatgttttattaaaacaaaagggacaacctattaaataattgccaatgggaaaaagtgtgtgcatgcattttgtgtatgtactcgcacatctagcaataccaggatttgtagaatgtgtgttttttttttcaaacacacgttaaaatagcttatgttgcatataccggataaagcacgcttgttattacgaaacgatagaattatgaagcttaaattttatcctaaaaagtgatgtgaaaaagtatgttttaagagcatgcagggcctataatggtaaatgttgaattaaaaacttacagtgctggcgaacagggacttagcgtgcctaggaactcaagtgcaaaaacaaaacaacatccacccccaaaaattgccctttttttttttcgaagtttgcagttatactattctgtgacttaaaagggaggggaaagaaagtatgcaattcagatttcgcccccaacgcaaaactaccgaaacgaaaatctccccaggaaaatgtggcttgatttatagacgaaaaagaaaaatattgttcctcttggcaaagtctttttgcatcacgtgtatttgcagcctagtataaacttgctttgccgtgtgtggatgtgtgagtgagagggaacgagagtaagagaaagaaagaagtgaggggatgtaaactcgaataaatttcaaagtgcctccgagggatgcaacgggcaaaaactgaactgttcaggcttcagattgtaactgacgatctgaggaaaaatgaggtgctcgatgaattttcgtttgtattttttggcgaggcgggggaggtgttgagattttttttttttcccctcggggtgggtgcgagggggatgcatcctagcctgcccgacccggagcaagtcgcgtctccccgccggagcccccccacccatttctttgctgaacttgcaattccgtgcgcctcggcgtgtttccccctccccccttccctccgtcccctcccctccccggagaagagagttggtgttaagagtcagggatcttggctgtgtgtctgcggatctgtagtggcggcggcggcggcggcggcggggaggcagcaggcgcgggagcgggcgcaggagcaggcggcggcggtggcggcggcggttagacatgaacgccgcctcggcgccggcggtgcacggagagccccttctcgcgcgcgggcggtaggtaccggcgcctgcggggctcggcggggcggaggcgcccggcggcgcggggttcgggctcggcggccccgcacgcggctccgcgcc

SEQ ID NO: 44 - DNA, 20 bp, synthetic construct
  source 1..20: mol_type = other DNA
ctaggcgctctgtttaccac

SEQ ID NO: 45 - DNA, 40 bp, synthetic construct
  source 1..40: mol_type = other DNA
acccttctccggccgctgacgtggtaaacagagcgcctag

SEQ ID NO: 46 - DNA, 110 bp, synthetic construct
  source 1..110: mol_type = other DNA
tccgctctggggcgcgcacaccctcgcccgcacttctcccctcgtccggcacggactgaccgcacacgaataacacatccgaccagagctgtggtaaacagagcgcctag

SEQ ID NO: 47 - DNA, 611 bp, synthetic construct
  source 1..611: mol_type = other DNA
  misc_difference 1..10: note = Cloning site
  misc_difference 602..611: note = Cloning site
  misc_difference 11..351: note = U7 promoter
  misc_difference 352..371: note = Regulatory element tail
  misc_difference 372..391: note = binding sequence
  misc_difference 392..430: note = sm OPT sequence
  misc_difference 431..601: note = U7 terminator
ttttggtacccccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcacccttctccggccgctgacgtggtaaacagagcgcctagaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggggtacctttt

SEQ ID NO: 48 - DNA, 681 bp, synthetic construct
  source 1..681: mol_type = other DNA
  misc_difference 1..10: note = Cloning site
  misc_difference 672..681: note = Cloning site
  misc_difference 11..351: note = U7 promoter
  misc_difference 352..441: note = Regulatory element tail
  misc_difference 442..461: note = Binding sequence
  misc_difference 462..500: note = sm OPT sequence
  misc_difference 501..671: note = U7 terminator
ttttggtacccccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgctccgctctggggcgcgcacaccctcgcccgcacttctcccctcgtccggcacggactgaccgcacacgaataacacatccgaccagagctgtggtaaacagagcgcctagaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggggtacctttt

SEQ ID NO: 49 - DNA, 5729 bp, synthetic construct
  source 1..5729: mol_type = other DNA
  misc_difference 662..767: note = mutated ITR
  misc_difference 808..1148: note = U7_Promoter
  misc_difference 1149..1168: note = Regulatory Element A
  misc_difference 1169..1188: note = Binding Sequence 1
  misc_difference 1189..1227: note = U7_SmOPT_RNA
  misc_difference 1228..1398: note = U7_3'end
  misc_difference 1405..2674: note = Stuffer
  misc_difference 2952..3092: note = ITR
  misc_difference 3926..4786: note = amp r
  misc_difference 4940..5561: note = pBR322 ori
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcacccttctccggccgctgacagacggggtttcggcatgttaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggggtacccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcaggggggggggggcgctgaggtctgcctcgtgaagaaggtgttgctgactcataccaggcctgaatcgccccatcatccagccagaaagtgagggagccacggttgatgagagctttgttgtaggtggaccagtagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcttatcgataccgtcgactagagctcgctgatcagtggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 50 - DNA, 5729 bp, synthetic construct
  source 1..5729: mol_type = other DNA
  misc_difference 662..767: note = mutated ITR
  misc_difference 808..1148: note = U7_Promoter
  misc_difference 1149..1168: note = Regulatory Element A
  misc_difference 1169..1188: note = Binding Sequence 2
  misc_difference 1189..1227: note = U7_SmOPT_RNA
  misc_difference 1228..1398: note = U7_3'end
  misc_difference 1405..2674: note = Stuffer
  misc_difference 2952..3092: note = ITR
  misc_difference 3926..4786: note = amp r
  misc_difference 4940..5561: note = pBR322 ori
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcacccttctccggccgctgacattgtgtaggctggtctcgaaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggggtacccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcaggggggggggggcgctgaggtctgcctcgtgaagaaggtgttgctgactcataccaggcctgaatcgccccatcatccagccagaaagtgagggagccacggttgatgagagctttgttgtaggtggaccagtagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcttatcgataccgtcgactagagctcgctgatcagtggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 51 - DNA, 5729 bp, synthetic construct
  source 1..5729: mol_type = other DNA
  misc_difference 662..767: note = mutated ITR
  misc_difference 808..1148: note = U7_Promoter
  misc_difference 1149..1168: note = Regulatory Element A
  misc_difference 1169..1188: note = Binding Sequence 3
  misc_difference 1189..1227: note = U7_SmOPT_RNA
  misc_difference 1228..1398: note = U7_3'end
  misc_difference 1405..2674: note = Stuffer
  misc_difference 2952..3092: note = ITR
  misc_difference 3926..4786: note = amp r
  misc_difference 4940..5561: note = pBR322 ori
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcacccttctccggccgctgacggggcaggaaggaaggcagaaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggggtacccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcaggggggggggggcgctgaggtctgcctcgtgaagaaggtgttgctgactcataccaggcctgaatcgccccatcatccagccagaaagtgagggagccacggttgatgagagctttgttgtaggtggaccagtagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcttatcgataccgtcgactagagctcgctgatcagtggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 52 - DNA, 5799 bp, synthetic construct
  source 1..5799: mol_type = other DNA
  misc_difference 662..767: note = mutated ITR
  misc_difference 808..1148: note = U7_Promoter
  misc_difference 1149..1238: note = Regulatory Element B
  misc_difference 1239..1258: note = Binding Sequence 1
  misc_difference 1259..1297: note = U7_SmOPT_RNA
  misc_difference 1298..1468: note = U7_3'end
  misc_difference 1475..2744: note = Stuffer
  misc_difference 3022..3162: note = ITR
  misc_difference 3996..4856: note = amp r
  misc_difference 5010..5631: note = pBR322 ori
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgctccgctctggggcgcgcacaccctcgcccgcacttctcccctcgtccggcacggactgaccgcacacgaataacacatccgaccagagctagacggggtttcggcatgttaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggggtacccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcaggggggggggggcgctgaggtctgcctcgtgaagaaggtgttgctgactcataccaggcctgaatcgccccatcatccagccagaaagtgagggagccacggttgatgagagctttgttgtaggtggaccagtagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcttatcgataccgtcgactagagctcgctgatcagtggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 53 - DNA, 5799 bp, synthetic construct
  source 1..5799: mol_type = other DNA
  misc_difference 662..767: note = mutated ITR
  misc_difference 808..1148: note = U7_Promoter
  misc_difference 1149..1238: note = Regulatory Element B
  misc_difference 1239..1258: note = Binding Sequence 2
  misc_difference 1259..1297: note = U7_SmOPT_RNA
  misc_difference 1298..1468: note = U7_3'end
  misc_difference 1475..2744: note = Stuffer
  misc_difference 3022..3162: note = ITR
  misc_difference 3996..4856: note = amp r
  misc_difference 5010..5631: note = pBR322 ori
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgctccgctctggggcgcgcacaccctcgcccgcacttctcccctcgtccggcacggactgaccgcacacgaataacacatccgaccagagctattgtgtaggctggtctcgaaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggggtacccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcaggggggggggggcgctgaggtctgcctcgtgaagaaggtgttgctgactcataccaggcctgaatcgccccatcatccagccagaaagtgagggagccacggttgatgagagctttgttgtaggtggaccagtagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcttatcgataccgtcgactagagctcgctgatcagtggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 54 - DNA, 5799 bp, synthetic construct
  source 1..5799: mol_type = other DNA
  misc_difference 662..767: note = mutated ITR
  misc_difference 808..1148: note = U7_Promoter
  misc_difference 1149..1238: note = Regulatory Element B
  misc_difference 1239..1258: note = Binding Sequence 3
  misc_difference 1259..1297: note = U7_SmOPT_RNA
  misc_difference 1298..1468: note = U7_3'end
  misc_difference 1475..2744: note = Stuffer
  misc_difference 3022..3162: note = ITR
  misc_difference 3996..4856: note = amp r
  misc_difference 5010..5631: note = pBR322 ori
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgctccgctctggggcgcgcacaccctcgcccgcacttctcccctcgtccggcacggactgaccgcacacgaataacacatccgaccagagctggggcaggaaggaaggcagaaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggggtacccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcaggggggggggggcgctgaggtctgcctcgtgaagaaggtgttgctgactcataccaggcctgaatcgccccatcatccagccagaaagtgagggagccacggttgatgagagctttgttgtaggtggaccagtagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcttatcgataccgtcgactagagctcgctgatcagtggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 55 - DNA, 5729 bp, synthetic construct
  source 1..5729: mol_type = other DNA
  misc_difference 662..767: note = mutated ITR
  misc_difference complement(808..978): note = U7 3' end
  misc_difference complement(979..1017): note = U7 Smopt
  misc_difference complement(1018..1037): note = Binding sequence
  misc_difference complement(1038..1057): note = Regulatory Element A
  misc_difference complement(1058..1398): note = U7 promoter
  misc_difference 1405..2674: note = Stuffer
  misc_difference 2952..3092: note = ITR
  misc_difference 3926..4786: note = amp r
  misc_difference 4940..5561: note = pBR322 ori
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccccagaggaggcagaaaggaagcgggaccgcacgaggcggaagtctcagagcgtggctctagcacatacgcgtttcctaggaaaccagagaaggatcaaagcccctctcacacaccggggagcggggaagagaactgttttgctttcattgtagaccagttaacttgggaggggttttccgaccgaagtcagaaaacctgctccaaaaattctaggcgctctgtttaccacacccttctccggccgctgacgcggaaaggcctctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttaaaaaaaaaaaaagagcttcgcgagattaaaggcttggagctaaagccgaagtgaatcggacttaagtagtggcaggcgatgtgggggtacccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcaggggggggggggcgctgaggtctgcctcgtgaagaaggtgttgctgactcataccaggcctgaatcgccccatcatccagccagaaagtgagggagccacggttgatgagagctttgttgtaggtggaccagtagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcttatcgataccgtcgactagagctcgctgatcagtggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 56 - DNA, 5799 bp, synthetic construct
  source 1..5799: mol_type = other DNA
  misc_difference 662..767: note = mutated ITR
  misc_difference complement(808..978): note = U7 3' end
  misc_difference complement(979..1017): note = U7 Smopt
  misc_difference complement(1018..1037): note = Binding sequence
  misc_difference complement(1038..1127): note = Regulatory element B
  misc_difference complement(1128..1468): note = U7 promoter
  misc_difference 1475..2744: note = Stuffer
  misc_difference 3022..3162: note = ITR
  misc_difference 3996..4856: note = amp r
  misc_difference 5010..5631: note = pBR322 ori
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccccagaggaggcagaaaggaagcgggaccgcacgaggcggaagtctcagagcgtggctctagcacatacgcgtttcctaggaaaccagagaaggatcaaagcccctctcacacaccggggagcggggaagagaactgttttgctttcattgtagaccagttaacttgggaggggttttccgaccgaagtcagaaaacctgctccaaaaattctaggcgctctgtttaccacagctctggtcggatgtgttattcgtgtgcggtcagtccgtgccggacgaggggagaagtgcgggcgagggtgtgcgcgccccagagcggagcggaaaggcctctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttaaaaaaaaaaaaagagcttcgcgagattaaaggcttggagctaaagccgaagtgaatcggacttaagtagtggcaggcgatgtgggggtacccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcaggggggggggggcgctgaggtctgcctcgtgaagaaggtgttgctgactcataccaggcctgaatcgccccatcatccagccagaaagtgagggagccacggttgatgagagctttgttgtaggtggaccagtagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcttatcgataccgtcgactagagctcgctgatcagtggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

(19) United States
(12) Patent Application Publication
(10) Pub. No.: US 2026/0218187 A1
(43) Pub. Date: Jul. 30, 2026

(54) MATERIALS AND METHODS FOR MODULATING EXPRESSION

(71) Applicant: RESEARCH INSTITUTE AT NATIONWIDE CHILDREN'S HOSPITAL, Columbus, OH (US)

(72) Inventors: Kathrin Meyer, Columbus, OH (US); Shibi Likhite, Columbus, OH (US); Samantha Lynn Powers, Columbus, OH (US)

(21) Appl. No.: 19/148,798

(22) PCT Filed: Jan. 18, 2024
(86) PCT No.: PCT/US2024/011984

§ 371 Date: Jul. 17, 2025

Related U.S. Application Data
(60) Provisional application No. 63/440,060, filed on Jan. 19, 2023.

(51) Int. Cl. C12N 15/113; A61P 21/00; A61P 25/00; C12N 15/86

(52) U.S. Cl. CPC C12N 15/113; A61P 21/00; A61P 25/00; C12N 15/86; C12N 2310/531; C12N 2750/14143

(57) ABSTRACT

The present disclosure relates to methods of modulating transcription of target genes using transcription modulators termed RNA-based transcription modulators or “RPMs”. The RPMs attract transcription regulators to the genes.

### CROSS-REFERENCE TO RELATED APPLICATIONS

This application claims priority to U.S. Provisional Patent Application No. 63/440,060, filed on Jan. 19, 2023, which is incorporated by reference herein in its entirety.

### INCORPORATION BY REFERENCE OF THE SEQUENCE LISTING

This application contains, as a separate part of disclosure, a Sequence Listing in computer-readable form (Filename: 56269_Seqlisting.xml; Size: 151,499 bytes; created: Jan. 18, 2024) which is incorporated by reference herein in its entirety.

### FIELD

The present disclosure relates to methods of modulating transcription of target genes using RNA-based promoter modulators (RPMs). The RPMs attract transcription regulators to the genes.

### BACKGROUND

The U7 small nuclear RNA was initially identified as a component of the U7 snRNP, a ribonucleoprotein complex specializing in processing histone mRNA.

Since its initial discovery, the U7 small nuclear RNA has been modified for use in gene therapy via transcript splicing modification in spinal muscular atrophy, amyotrophic lateral sclerosis, Duchenne Muscular Dystrophy, and β-thalassemia/Hemoglobin E disorder.

There remains a need in the art for products and methods for regulating transcription and treating conditions resulting from aberrant transcription.

### SUMMARY

The disclosure herein provides RNAs (referred to as “RPMs” herein) engineered to alter transcription of target genes by different mechanisms than splicing modification. The RPMs comprise (a) a DNA-binding component that binds a regulatory region including, but not limited to, the promoter region of a gene of interest and (b) a tail that contains regulatory elements that attract proteins that increase or reduce transcription including, but not limited to, transcription activators, transcription repressors, DNA-modifying enzymes, and RNA polymerase II.

While incorporating some U7 elements, the RPMs differ from U7 small nuclear RNAs in a number of ways. U7 small nuclear RNAs bind RNA transcripts, while RPMs are engineered to bind genomic DNA. This results in a difference in the orientation of some components of the RPMs versus U7 small nuclear RNAs. In one example configuration, the binding sequence of the RPMs binds the 5′ DNA strand (sense strand) of a gene. The regulatory element tail of the RPMs is oriented so that the proteins binding the regulatory element are also in contact with the 5′ DNA strand of the promoter. Furthermore, RPMs are assembled so that the sm OPT consensus sequence (capable of binding sm and sm-like proteins) and a structured RNA hairpin are positioned downstream of the promoter so that these components do not interfere with transcription factor- or RNA polymerase II-binding and transcription. Therefore, the binding sequence of the RPMs is in the reverse complement orientation of the DNA 5′ binding site, and the regulatory element tail is in the reverse orientation of the endogenous regulatory element (or transcription factor binding site). Alternatively, the RPMs can target the 3′ DNA strand (antisense/non-coding strand) of a gene. The sm OPT and the hairpin are important for stabilizing the U7 small nuclear RNA. The binding sequence and regulatory element tail of the RPMs may be adapted to tailor the RPMs for different target genes and different effects on transcription, respectively.

Thus, the disclosure provides RPMs comprising: (a) a regulatory element tail; (b) a binding sequence; and (c) an Sm OPT consensus sequence and hairpin. As one example, the orientation of (a), (b) and (c) in the RPM is 5′ to 3′ and the (b) binding sequence binds the sense strand of a target gene promoter.

The disclosure provides example SMN2 RPMs comprising: (a) Regulatory Element A Tail encoded by ACCCTTCTCCGGCCGCTGAC (SEQ ID NO: 1) or Regulatory Element B Tail encoded by TCCGCTCTGGGGCGCGCACACCCTCGCCCGCACTTCTCCCCTCGTCCGGCACGGACT GACCGCACACGAATAACACATCCGACCAGAGCT (SEQ ID NO: 2); and (b) Binding Sequence 1 encoded by AGACGGGGTTTCGGCATGTT (SEQ ID NO: 3), Binding Sequence 2 encoded by ATTGTGTAGGCTGGTCTGA (SEQ ID NO: 4), or Binding Sequence 3 encoded by GGGGCAGGAAGGAAGGCAGA (SEQ ID NO: 5).

The disclosure provides example TCF4 RPMs comprising: (a) Regulatory Element A Tail encoded by ACCCTTCTCCGGCCGCTGAC (SEQ ID NO: 1) or Regulatory Element B Tail encoded by TCCGCTCTGGGGCGCGCACACCCTCGCCCGCACTTCTCCCCTCGTCCGGCACGGACT GACCGCACACGAATAACACATCCGACCAGAGCT (SEQ ID NO: 2); and (b) Binding Sequence 1 encoded by GTGGTAAACAGAGCGCCTAG (SEQ ID NO: 6).

The disclosure provides nucleic acids encoding RPMs, the nucleic acid comprising: (a) a DNA encoding (i) an RPM of claim 1 or 2, (ii) an SMN2 RPM of claim 3, or (iii) a TCF4 RPM of claim 4; (b) a DNA construct of FIG. 7A or 7B; or (c) a DNA at least 90% identical to the DNA of (a) or DNA construct of (b). The nucleic acid of (a) can further comprise a promoter driving expression of the RPM. The promoter can be a U7 promoter. The promoter can be, as other examples, a U6 promoter, a tRNA promoter, a H1 promoter, a minimal CMV promoter, a T7 promoter, an EF1-alpha promoter, a Minimal EF1-alpha promoter.

The disclosure provides recombinant adeno-associated virus (rAAV) comprising the nucleic acid provided herein. The rAAV can be a single-stranded AAV (ssAAV) or a self-complementary AAV (scAAV). The rAAV can be a rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh.10, AAV-B1, MyoAAV, MYOAAV1A, SLB101, M1 and M3, or their derivatives.

The disclosure provides compositions comprising (a) a nucleic acid provided herein or a rAAV provided herein and a pharmaceutically acceptable carrier.

The disclosure provides methods of modulating expression of a target gene in a cell comprising delivering an RPM provided herein to the cell.

The disclosure provides methods of modulating expression of a target gene in a cell comprising contacting the cell with: (a) a nucleic acid provided herein; (b) a rAAV provided herein, or (c) a composition provided herein. The methods can increase expression. The methods can decrease expression.

The disclosure provides nucleic acids, rAAV and compositions, wherein the nucleic acids, rAAV and compositions are formulated for intramuscular injection, intrathecal injection, transdermal transport or injection into the blood stream.

The disclosure provides methods of treating spinal muscular atrophy in a subject comprising delivering a SMN2 RPM to the subject.

The disclosure provides methods of treating Pitt Hopkins Syndrome in a subject comprising delivering a TCF4 RPM to the subject.

### BRIEF DESCRIPTION OF THE DRAWINGS

FIG. 1 (A) is a schematic showing binding of an RPM which is the reverse complement of the sense strand of the target DNA and showing the 5′ to 3′ arrangement of the components of the RPM. The (A) RPM orients so that the regulatory element tail and binding sequence are on the 3′ side of the promoter in the target DNA. FIG. 1 (B) shows an alternative orientation for the RPM which is the reverse complement of the antisense/non-coding strand of the target DNA.

FIG. 2 shows binding motifs in the SMN2 promoter outside of previously identified binding sites for transcription factors (modified from Monami et al. 1999) (A). Sequences encoding SMN2 promoter binding sequences for an RPM are shown in sense and reverse complement orientation (B).

FIG. 3 shows the design of RPM regulatory elements and SMN2 binding sequences in SMN2 RPM constructs exemplified herein. SMN2 RPM constructs A1-2 feature regulatory element A with binding sequences 1-3, respectively. SMN2 RPM constructs B1-3 feature regulatory element B with binding sequences 1-3, respectively.

FIG. 4 shows TATA box and enhancer elements within the 2000 bp region upstream of Exon of TCF4.

FIG. 5 shows the targeted location of a TCF4 promoter binding sequence 1692 bp upstream of TCF4-201 EXON1 (A). The TCF4 promoter binding sequence is shown in sense and reverse complement orientation (B).

FIG. 6 shows the design of RPM regulatory elements and TCF4 binding sequences in TCF4 RPM constructs exemplified herein. TCF4 RPM Construct A1 features regulatory element A with binding sequence 1. TCF4 RPM Construct B1 features regulatory element B with binding sequence 1.

FIG. 7 shows TCF4 RPM DNA constructs in which cloning sites are shown in uppercase type at the ends of each construct. Then in order after the cloning site at the beginning of each construct, the U7 promoter is shown in in lowercase type, the sequence encoding the regulatory element tail is shown in uppercase type, the sequence encoding the binding sequence is shown in underlined uppercase type, the sequence encoding the sm OPT sequence is shown in lowercase type and the sequence encoding the U7 terminator is shaded.

FIG. 8 shows U7.TCF4 Plasmid Constructs upregulate TCF4 Expression in HEK293 cells. U7 expression analyzed by PCR and gel electrophoresis demonstrates presence of U7 transcript in HEK-293 cells transfected with B1.U7.TCF4 plasmid constructs, 72 hours post transfection (A). qPCR demonstrates up to two-fold increase in total TCF4 mRNA expression with B1.U7.TCF4 plasmid constructs in 293 cells 72 hrs post transfection. The primer pair amplifies all TCF4 isoforms (B). Western blot analysis demonstrates up to 1.5-fold increase in total TCF4 protein with B1.U7.TCF4 plasmid constructs in 293 cells 72 hrs post transfection (C).

FIG. 9 shows AAV production plasmids and the sequences of the plasmids: (A) pscAAV.A1-U7.SMN (5′-3′), (B) pscAAV.A2-U7.SMN (5′-3′), (C) pscAAV.A3-U7.SMN (5′-3′), (D) pscAAV.B1-U7.SMN (5′-3′), (E) pscAAV.B2-U7.SMN (5′-3′), (F) pscAAV.B3-U7.SMN (5′-3′), (G) pscAAV.A1-U7.TCF4 (3′-5′), and (H) pscAAV.B1-U7.TCF4 (3′-5′).

FIG. 10 shows elevated U7 PRM expression and concurrent increase in TCF4 expression in AAV9.U7.TCF4-treated hTcf4-H2B-EGFP-Nluc MEF cell lines. HOMO MEFs are mouse embryonic fibroblasts homozygous for humanized TCF4 promoter-driven expression. The cells were subjected to insulin mediated transduction with AAV9.RFP or AAV9.U7.TCF4 and analyzed 72 hours post transduction for U7 expression and mouse TCF4 expression.

FIG. 11 shows an increase in TCF4 mRNA and protein expression after scAAV9.B1.U7.TCF4 treatment in an in vitro disease model of induced astrocytes from Pitt-Hopkin's disease syndrome (PTHS) patients.

FIG. 12 shows treatment of PTHS patient induced astrocytes with scAAV9.B1.U7.TCF4 results in significant rescue of neuron survival in another in vitro disease model, coculture of PTHS patient-derived induced astrocytes with mouse HB9-GFP neurons.

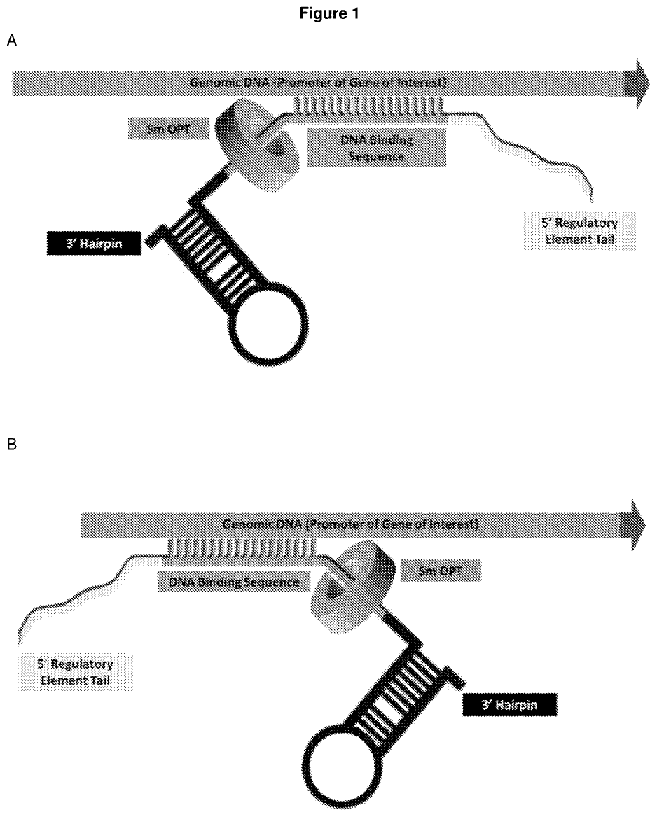

### DETAILED DESCRIPTION

In the RPM approach, an RPM finds a target site (variously referred to as a target motif, binding site or binding motif herein) in the genome and attracts an endogenous or exogenous intracellular protein to modify gene expression at that site. The approach utilizes all mammalian-derived components, can make use of endogenous proteins, is highly specific, stable, and safe.

### RPMs

The disclosure provides RPMs comprising: (a) a regulatory element tail; (b) a binding sequence; and (c) an Sm OPT consensus sequence and hairpin. As one example, the orientation of (a), (b) and (c) in the RPM is 5′ to 3′ and the (b) binding sequence binds the sense strand of a target gene promoter.

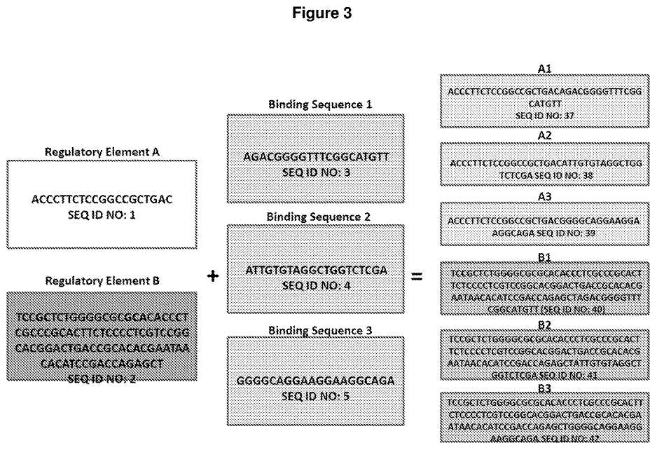

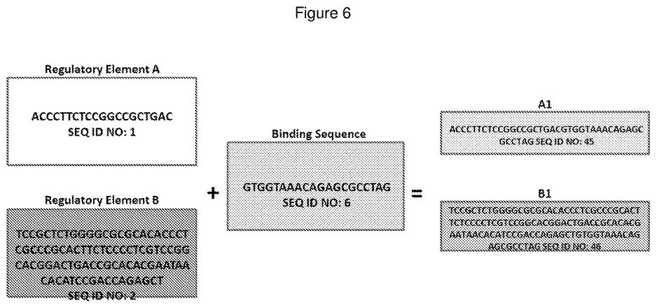

FIGS. 3 and 6 also set out examples of sequences encoding regulatory element tails that can be used in RPMs as described herein.

FIGS. 3 and 6 set out examples of sequences encoding binding sequences that can be used in RPMs as described herein.

The disclosure provides example SMN2 RPMs comprising: (a) Regulatory Element A Tail encoded by ACCCTTCTCCGGCCGCTGAC (SEQ ID NO: 1) or Regulatory Element B Tail encoded by TCCGCTCTGGGGCGCGCACACCCTCGCCCGCACTTCTCCCCTCGTCCGGCACGGACT GACCGCACACGAATAACACATCCGACCAGAGCT (SEQ ID NO: 2); and (b) Binding Sequence 1 encoded by AGACGGGGTTTCGGCATGTT (SEQ ID NO: 3), Binding Sequence 2 encoded by ATTGTGTAGGCTGGTCTGA (SEQ ID NO: 4), or Binding Sequence 3 encoded by GGGGCAGGAAGGAAGGCAGA (SEQ ID NO: 5).

The disclosure provides example TCF4 RPMs comprising: (a) Regulatory Element A Tail encoded by ACCCTTCTCCGGCCGCTGAC (SEQ ID NO: 1) or Regulatory Element B Tail encoded by TCCGCTCTGGGGCGCGCACACCCTCGCCCGCACTTCTCCCCTCGTCCGGCACGGACT GACCGCACACGAATAACACATCCGACCAGAGCT (SEQ ID NO: 2); and (b) Binding Sequence 1 encoded by GTGGTAAACAGAGCGCCTAG (SEQ ID NO: 6).

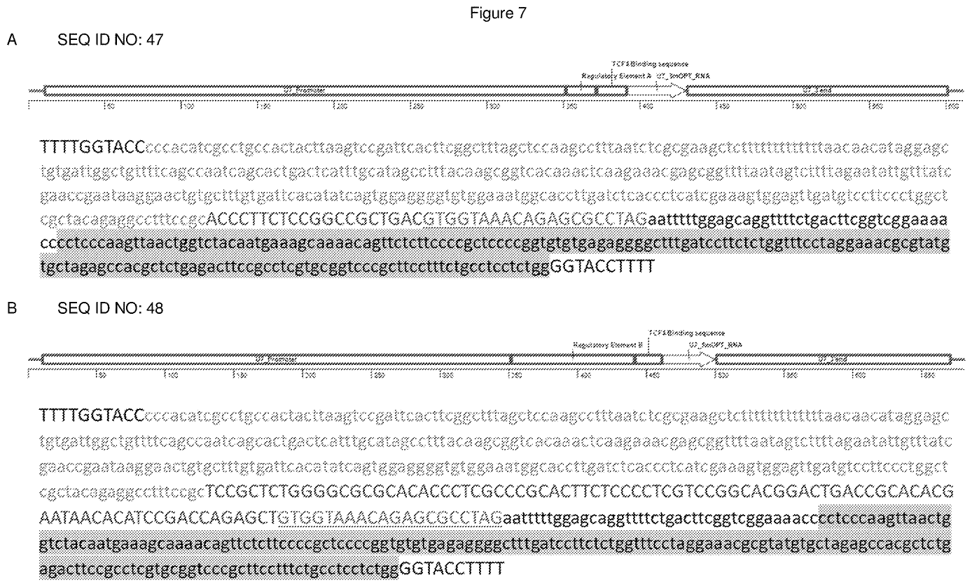

The disclosure provides nucleic acids encoding RPMs, the nucleic acid comprising: (a) a DNA encoding (i) an RPM of claim 1 or 2, (ii) an SMN2 RPM of claim 3, or (iii) a TCF4 RPM of claim 4; (b) a DNA construct of FIG. 7A or 7B; or (c) a DNA at least 90% identical to the DNA of (a) or DNA construct of (b). The nucleic acid of (a) can further comprise a promoter driving expression of the RPM. The promoter can be a U7 promoter. The promoter can be, as other examples, a U6 promoter, a tRNA promoter, a H1 promoter, a minimal CMV promoter, a T7 promoter, an EF1-alpha promoter, a Minimal EF1-alpha promoter. A U7 expression cassette can be cloned in a vector in forward or reverse orientation.

### Delivery Vectors

Vectors for delivering polynucleotides encoding RPMs to cells are provided herein. Such vectors include, but are not limited to, viral vectors, such as adeno-associated virus, adenovirus, retrovirus, lentivirus, equine-associated virus, alphavirus, pox virus, herpes virus, herpes simplex virus, polio virus, sindbis virus, vaccinia virus or a synthetic virus (e.g., a chimeric virus, mosaic virus, pseudotyped virus, and/or a virus that contains a foreign protein, synthetic polymer, nanoparticle, or small molecule).

Recombinant adeno-associated virus are provided to deliver DNA encoding an RPM to a cell.

Adeno-associated virus (AAV) is a replication-deficient parvovirus, the single-stranded DNA genome of which is about 4.7 kb in length including two 145 nucleotide inverted terminal repeats (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AAVs may also be referred to by the clade. For example, AAV9 sequences are referred to as “clade F” sequences (Gao et al., J. Virol., 78: 6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). Anc80 is an AAV vector that is of AAV1, AAV2, AAV8 and AAV9. The sequence of Anc80 is provided in Zinn et al., Cell Reports 12: 1056-1068, 2015 and Vandenberghe et al, PCT/US2014/060163, both of which are incorporated by reference herein, in their entirety and GenBank Accession Nos. KT235804-KT235812.

Cis-acting sequences directing viral DNA replication, encapsidation/packaging, and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter, and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (560 to 650° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to recombinant, infectious, encapsulated virus or viral particles. The terms “rAAV,” “rAAV virus,” and “rAAV viral particle” are used interchangeably herein.

The term “rAAV genome” refers to a polynucleotide sequence that is derived from a native AAV genome that has been modified. rAAV genomes are provided that have been modified to remove the native AAV cap and rep genes. The rAAV genomes comprise at least one or both endogenous 5′ and 3′ inverted terminal repeats (ITRs). The rAAV genome can comprise ITRs from an AAV serotype that is different from the AAV serotype from which the AAV genome was derived.

rAAV genomes comprising a transgene flanked at the 5′ and 3′ ends by AAV ITRs are provided herein.

Transgenes provided herein include, but are not limited to, a transgene comprising an RPM DNA, or comprising a polynucleotide 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the RPM DNA.

Transgenes provided herein can include an RPM polynucleotide that encodes an RPM and that hybridizes under stringent conditions to another RPM polynucleotide provided herein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. See, for example, Sambrook et al., Molecular Cloning: A Laboratory Manual, 2nd Ed., Cold Spring Harbor Laboratory, (Cold Spring Harbor, N.Y. 1989).

Examples of promoters contemplated herein include, but are not limited to, the U7 promoter, simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as the actin promoter, the myosin promoter, the elongation factor-1a promoter, the hemoglobin promoter, and the creatine kinase promoter. Additionally provided herein are promoters at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to one of those promoters which possess transcription promoting activity.

Examples of transcription control elements are tissue specific control elements, for example, promoters that allow expression specifically within neurons or specifically within astrocytes. Examples include neuron specific enolase and astrocyte-specific glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

“Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g., wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes Anc80, Anc80L65, AAV-1, AAV-2, AAV-3, AAV-4, AAV-5, AAV-6, AAV-7, AAV7mb, AAV-8, AAV-9, AAV-10, AAV-RH10, AAV-11, AAV-12, AAV-13, AAV rh.74, AAV-B1, MyoAAV, MYOAAV1A, SLB101, M1 and M3, and their derivatives. As noted above, the nucleotide sequences of the genomes of various AAV serotypes are known in the art. rAAV with capsid mutations, are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. AAV capsid proteins may be modified to enhance delivery of the recombinant rAAV. Modifications to capsid proteins are generally known in the art. See, for example, US 2005/0053922 and US 2009/0202490. Capsid proteins of a rAAV may be modified so that the rAAV is targeted to a particular target tissue of interest such as neurons. See, for example, WO 02/053703.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing (Samulski et al., 1982, Proc. Natl. Acad. S6. USA, 79:2077-2081), addition of synthetic linkers containing restriction endonuclease cleavage sites [Laughlin et al., Gene, 23:65-73 (1983)] or by direct, blunt-end ligation [Senapathy & Carter, J. Biol. Chem., 259:4661-4666 (1984)]. The packaging cell line may then be infected with a helper virus such as adenovirus. The advantages of this method are that the cells are selectable and are suitable for large-scale production of rAAV. Other non-limiting examples of suitable methods employ adenovirus or baculovirus rather than plasmids to introduce rAAV genomes and/or rep and cap genes into packaging cells.

General principles of rAAV particle production are reviewed in, for example, Carter, Current Opinions in Biotechnology, 1533-1539 (1992); and Muzyczka, Curr. Topics in Microbial. and Immunol., 158:97-129 (1992). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-1250 (1995); Paul et al., Human Gene Therapy, 4:609-615 (1993); Clark et al., Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. A rAAV with a single-stranded genome is referred to herein as an ssAAV.

The rAAV may be purified by methods standard in the art such as by column chromatography or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

### Compositions

Compositions comprising rAAV are also provided. Compositions comprise a rAAV encoding an RPM. Compositions may include two or more rAAV encoding different RPMs of interest.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate e.g., phosphate-buffered saline (PBS), citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound or contrast agent such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgl/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in and 1×PBS and 0.001% Pluronic F68. Simply resuspending a rAAV in phosphate buffered saline has been demonstrated to be sufficient to provide a vehicle useful for muscle tissue expression.

For CSF delivery including but not limited to intrathecal delivery, the viral vector can be mixed with a contrast agent (Omnipaque or similar). For example, the compositions may comprise a non-ionic, low-osmolar contrast agent including, but not limited to, iobitridol, iohexol, iomeprol, iopamidol, iopentol, iopromide, ioversol, ioxilan, or combinations thereof.

Dosages may be expressed in units of viral genomes (vg). Dosages contemplated herein include about 1×10⁷ vg, about 1×10⁸ vg, about 1×10⁹ vg, about 5×10⁹ vg, about 6×10⁹ vg, about 7×10⁹ vg, about 8×10⁹ vg, about 9×10⁹ vg, about 1×10¹⁰ vg, about 2×10¹⁰ vg, about 3×10¹⁰ vg, about 4×10¹⁰ vg, about 5×10¹⁰ vg, about 1×10¹¹ vg, about 1.1×10¹¹ vg, about 1.2×10¹¹ vg, about 1.3×10¹¹ vg, about 1.2×10¹¹ vg, about 1.3×10¹¹ vg, about 1.4×10¹¹ vg, about 1.5×10¹¹ vg, about 1.6×10¹¹ vg, about 1.7×10¹¹ vg, about 1.8×10¹¹ vg, about 1.9×10¹¹ vg, about 2×10¹¹ vg, about 3×10¹¹ vg, about 4×10¹¹ vg, about 5×10¹¹ vg, about 1×10¹² vg, about 1×10¹³ vg, about 1.1×10¹³ vg, about 1.2×10¹³ vg, about 1.3×10¹³ vg, about 1.5×10¹³ vg, about 2×10¹³ vg, about 2.5×10¹³ vg, about 3×10¹³ vg, about 3.5×10¹³ vg, about 4×10¹³ vg, about 4.5×10¹³ vg, about 5×10¹³ vg, about 6×10¹³ vg, about 1×10⁴ vg, about 2×10⁴ vg, about 3×10¹⁴ vg, about 4×10¹⁴ vg, about 5×10¹⁴ vg, about 1×10¹⁵ vg, to about 1×10¹⁶ vg, or more total viral genomes. Dosages of about 1×10¹ vg to about 1×10¹⁰ vg, about 5×10¹ vg to about 5×10¹⁰ vg, about 1×10¹⁰ vg to about 1×10¹¹ vg, about 1×10¹¹ vg to about 1×10¹⁵ vg, about 1×10¹² vg to about 1×10¹⁵ vg, about 1×10¹² vg to about 1×10¹⁴ vg, about 1×10¹³ vg to about 6×10¹⁴ vg, and about 6×10¹³ vg to about 1.0×10¹⁴ vg, 2.0×10¹⁴ vg, 3.0×10¹⁴ vg, 5.0×10¹⁴ are also contemplated. One dose exemplified herein is 1.65×10¹¹ vg.

For example, CSF doses can range between about 1×10¹³ vg/patient to about 1×10¹⁵ vg/patient based on age groups. For example, intravenous delivery doses can range between 1×10¹³ vg/kilogram (kg) body weight and 2×10⁴ vg/kg.

### Methods of Treatment

The treatment methods provided herein comprise the step of administering an effective dose, or effective multiple doses, of a composition comprising a rAAV provided herein to a subject (e.g., an animal including, but not limited to, a human patient) in need thereof. If the dose is administered prior to development of symptoms of condition being treated, the administration is prophylactic. If the dose is administered after the development of symptoms, the administration is therapeutic. An effective dose is a dose that alleviates (eliminates or reduces) at least one symptom associated with the condition, that slows or prevents progression of the condition, that diminishes the extent of the condition, that results in remission (partial or total) of the condition, and/or that prolongs survival.

Treatment methods provided herein transduce target cells with one or more rAAV described herein. Transduction of cells with rAAV of the disclosure results in sustained expression of RPM encoded by the rAAV.

Administration of an effective dose of a nucleic acid, viral vector, or composition of the disclosure is contemplated by a route standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal route. An effective dose can be delivered by a systemic route of administration, i.e., systemic administration. Examples of systemic administration are enteral administration (absorption of the drug through the gastrointestinal tract) or parenteral administration (generally via injection, infusion, or implantation). An effective dose can be delivered by a combination of routes. An effective dose can comprise multiple administrations delivered in sequentially or simultaneously. Route(s) of administration and serotype(s) of AAV components of the rAAV (in particular, the AAV ITRs and capsid protein) are chosen and/or matched by those skilled in the art taking into account the condition or state of the disease or disorder being treated; the condition, state, or age of the subject; and the target cells/tissue(s) that are to express the transgene.

A rAAV viral particle comprising a transgene can be administered or delivered to the CSF of a subject by, for example, by intracerebroventricular injection, cisternal injection or lumbar intrathecal injection, or other injection method(s) accessing the CSF, or via intravenous delivery, or via a combination of such routes. Intrathecal administration refers to delivery into the space under the arachnoid membrane of the brain or spinal cord. Intrathecal administration to the brain in particular can be carried out by intracerebroventricular injection. Areas of the brain contemplated for delivery include, but are not limited to, the motor cortex, visual cortex, cerebellum and the brain stem.

For intrathecal administration, the subject can be held in the Trendelenburg position (head down position) after injection of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees.

### Kits

The disclosure also provides a kit comprising a nucleic acid, vector, or composition of the disclosure. The term “kit” means two or more components, one of which corresponds to a nucleic acid, vector, or composition of the disclosure, and the other which corresponds to a container, recipient, instructions, or otherwise.

The kit can comprise one or more recipients (such as vials, ampoules, containers, syringes, bottles, bags) of any appropriate shape, size and material containing the nucleic acid, vector, or composition of the disclosure in an appropriate dosage for administration (see above). The kit may additionally contain directions or instructions for use (e.g., in the form of a leaflet or instruction manual); means for administering the nucleic acid, vector, or composition, such as a syringe, pump, infuser or the like; means for reconstituting the nucleic acid, vector, or composition; and/or means for diluting the nucleic acid, vector, or composition.

The kit can comprise a label and/or instructions that describes use of the components provided in the kit. The kits also optionally comprise catheters, syringes or other delivering devices for the delivery of one or more of the compositions used in the methods described herein.

The disclosure also provides kits for a single dose or for multiple doses. For example, the disclosure provides kits containing single-chambered and multi-chambered pre-filled syringes.

### Other Terminology and Disclosure

Unless defined otherwise, all technical and scientific terms used herein have the same meaning as commonly understood by one of ordinary skill in the art to which this disclosure belongs. Any methods and materials similar or equivalent to those provided herein can also be used in the practice or testing of the present disclosure.

All publications mentioned herein are incorporated herein by reference to disclose and describe the methods and/or materials for the purpose for which the publications are cited.

As used herein and in the appended claims, the singular forms “a,” “and” and “the” include plural referents unless the context clearly dictates otherwise. Thus, for example, reference to “an antigen” includes a plurality of such antigens, and reference to “a cell” or “the cell” includes reference to one or more cells and equivalents thereof (e.g., plurality of cells) known to those skilled in the art, and so forth. Similarly, reference to “a compound” or “a composition” includes a plurality of such compounds or compositions, and refers to one or more compounds or compositions, respectively, unless the context clearly dictates otherwise.

It is further noted that the claims may be drafted to exclude any element, e.g., any optional element. As such, this statement is intended to serve as antecedent basis for use of such exclusive terminology as “solely,” “only” and the like in connection with the recitation of claim elements or use of a “negative” limitation.

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step.

The term “about” when referring to a number or a numerical range means that the number or numerical range referred to is an approximation within experimental variability (or within statistical experimental error), and thus the number or numerical range may vary between 1% and 15% of the stated number or numerical range.

Throughout this specification and the claims which follow, unless the context requires otherwise, the word “comprise”, and variations such as “comprises” and “comprising”, will be understood to mean the inclusion of a stated component or step, or group of components or steps, but not the exclusion of any other component or step or group of components or steps.

When used herein, “consisting of” excludes any element, step, or ingredient not specified in the claim element. When used herein, “consisting essentially of” does not exclude materials or steps that do not materially affect the basic and novel characteristics of the claim element.

The term “comprising” (and related terms such as “comprise” or “comprises” or “having” or “including”) is not intended to exclude that, for example, a composition of matter, composition, method, or process, or the like, provided herein, may “consist of” or “consist essentially of” the described features.

As will be apparent to those of skill in the art upon reading this disclosure, each of the compositions and methods provided and illustrated herein has discrete components and features which may be readily separated from or combined with the features of any of the other compositions and methods without departing from the scope or spirit of the present disclosure. Any recited method can be carried out in the order of events recited or in any other order which is logically possible. This disclosure is intended to provide support for all such combinations.

As used herein, “contemplated,” “may,” “may comprise,” “may be,” “can,” “can comprise” and “can be” all indicate something envisaged by the inventors that is functional and available as part of the subject matter provided.

### EXAMPLES

While the following examples describe specific embodiments, variations and modifications will occur to those skilled in the art. Accordingly, only such limitations as appear in the claims should be placed on the invention.

### Example 1

### Targeting Regulatory Elements in the Genome

### Forward Selection of Genomic Targets

A bioinformatics analysis was performed to specifically identify relatively small endogenous regulatory elements in the human genome utilizing the online database of enhancer elements EnhancerAtlas (www.enhanceratlas.org). The database includes targets predicted by: EP300 histone acetyltransferase binding sites, RNA Polymerase II binding sites, DNAse I hypersensitivity sites, and H3K4me1 and H3K27ac histone modification patterns, eRNA sequences, among others 7. EnhancerAtlas (www.enhanceratlas.org) contains predicted targets from multiple human tissues including several that are central nervous system (CNS) tissues: astrocytes, neurons derived from embryonic stem cells, fetal brain, fetal spinal cord, cerebellum, and retina [Gao et al., EnhancerAtlas: a resource for enhancer annotation and analysis in 105 human cell/tissue types. doi:10.1093/bioinformatics/btw495]. All of the predicted enhancers 100 bp or shorter were selected (Table 1).

TABLE 1
Predicted CNS Enhancers by Region and Size

| | | # of En- |
|---|---|---|
| | # of Enhancers | hancers </= |
| Tissue | Predicted | 100 bp |
| Embryonic Stem Cell- | 23,486 | 278 |
| Derived Neurons | | |
| Cerebellum | 17,103 | 548 |
| Retina | 7,439 | 222 |
| Fetal Spinal Cord | 7,953 | 89 |
| Fetal Brain | 49,924 | 408 |
| Astrocytes | 44,489 | 224 |

Of these, eleven were common to all the tissues and were chosen as pan-neuronal regulatory elements. Moreover, in the fetal spinal cord, there were several regulatory elements that were 40 bp or shorter, which, while not pan neuronal, were chosen as targets due to their very small size (Table 2).

TABLE 2
Selected Regulatory Elements for U7 Small Nuclear RNA Construct

| Pan-neuronal Regulatory Element | Spinal Cord Regulatory Element | Length (bp) |
|---|---|---|
| | chr15: 89164610-89164630 | 20 |
| | chr19: 1479140-1479160 | 20 |
| | chr20: 5591470-5591490 | 20 |
| | chr17: 2297310-2297340 | 30 |
| | chr19: 1383940-1383970 | 30 |
| | chr6: 15663010-15663040 | 30 |
| | chr20: 34360000-34360040 | 40 |
| chr5: 176943840-176943910 | | 70 |
| chr7: 140396640-140396710 | | 70 |
| chr1: 228290750-228290830 | | 80 |
| chr3: 149470080-149470160 | | 80 |
| chr5: 41870300-41870380 | | 80 |
| chr2: 74699600-74699690 | | 90 |
| chr2: 135809900-135809990 | | 90 |
| chr7: 44613340-44613430 | | 100 |
| chr8: 146277880-146277970 | | 100 |
| chr2: 74699380-74699480 | | 100 |
| chr6: 99872990-99873090 | | 100 |

Additionally, seven very small elements (20-40 bp) identified in spinal cord were chosen.

TABLE 3
Very Small Elements in Spinal Cord

chr15: 89164610-89164630
chr19: 1479140-1479160
chr20: 5591470-5591490
chr17: 2297310-2297340
chr19: 1383940-1383970
chr6: 15663010-15663040
chr20: 34360000-34360040

### Backward Selection of Genomic Targets

Other target sites in the genome were selected after preliminarily identification of seventy-three transcription factors expressed in the CNS. JASPAR software [Fornes et al, Nucleic Acids Res., 48: 87-92 (2019)] was used to find the consensus binding sequence for each of these transcription factors (Table 4). A secondary approach was to build synthetic regulatory elements around a binding motif for a transcription factor of interest [Fornes et al., JASPAR 2020: update of the open-access database of transcription factor binding profiles. Nucleic Acids Res., 48:87-92 (2019)].

TABLE 4
Common Binding Motifs of CNS Enriched Transcription Factors

| Transcription Factor | Common Binding Motif | Species |
|---|---|---|
| arnt2 | GTCACGTGC | Human |
| Arx | TTAATTAA | Mouse |
| bcl11b | GTGAACG(C/T) CTACAC (SEQ ID NO: 7) | Human |
| bcl6 | TTCCT(A/G)GAAAGC (SEQ ID NO: 8) | Mouse |
| bcl6 | GCTTTC(G/T)AGGAA (SEQ ID NO: 9) | Human |
| Bcl6 | TTCCT(A/G)(G/A)A(A/G/C)(A/G/T) | Mouse |
| | (SEQ ID NO: 10) | |
| Bcl6 | GCTTTC(G/T)AGGAA (SEQ ID NO: 11) | Human |
| COUP-TFI (NR2F1) | TGA(C/A)CTTTG(A/C/G)(A/C)C(C/T/A)(T/C/G) | Human |
| crx | (G/A)G(A/C)TTA(G/A) | Mouse |
| crx | GGGGATTA | Mouse |
| cux2 | T(A/G)ATC(G/A)AT(A/T/C) | Human |
| dlx1 | TAATTA | Mouse |
| dlx2 | (T/C)AATTA | Mouse |
| dlx5 | (C/T)AATTA | Human |
| dlx6 | (C/T)AATTA | Human |
| ebf3 | CCCAAGGGA | Human |
| egr3 | (A/C)CGCCC(A/C)CGCA | Human |
| EMX2 | TAATTA | Human |
| foxd1 | GTAAACA | Human |
| foxg1 | (G/A)TAAACA | Human |
| foxp2 | GTAAACA | Human |
| gbx1 | CTAATTA | Human |
| gli3 | GACCACCCAC (SEQ ID NO: 12) | Human |
| glis3 | (G/T)ACCCCCCAC(A/G)C(G/A)AAG | Human |
| | (SEQ ID NO: 13) | |
| Hes1 | CACGCG | Mouse |
| Hes5 | GGCACGTGCC (SEQ ID NO: 14) | Human |
| hlf | (G/A)TTAC(G/A)(C/T AAT | Human |
| hlf | TTA(C/T)(G/A)TAA(C/T) | Human |
| hlf | TTATG(C/T)AA(C/T) | Human |
| hlf | (G/A)TTAC(G/A)(C/T)(A/C)AT | Human |
| hoxa7 | (C/T)(A/C)ATTA | Human |
| hoxc8 | TAATTA | Human |
| irf9 | A(A/T)CGAAACCGAAACT (SEQ ID NO: 15) | Human |
| Isl1 | CCATTAG | Mouse |
| isl2 | CA(C/A)TTA | Human |
| klf5 | GCCCC(G/A/T)CCC(C/T/A) | Human |
| KLF5 | CCCC(G/A/T)CCC | Human |
| Lhx2 | CTAATTA | Human |
| Lhx2 | CCAATTA | Human |
| Lhx2 | ACTAATTAG | Mouse |
| lhx3 | AATTAATTAAT (SEQ ID NO: 16) | Mouse |
| lhx6 | TAATTA | Human |
| lmx1b | TAATTA | Human |
| Lmx1b | TTAATTA | Human |
| mef2a | CTATTTATAG (SEQ ID NO: 17) | Human |
| mef2a | CTAAAAATAG (SEQ ID NO: 18) | Human |
| mef2a | CTA(A/T)AAATAG | Human |
| mef2a | CTAAAAATAG (SEQ ID NO: 19) | Human |
| mef2d | CTA(T/A)AAATAG | Human |
| meis1 | CTGTCA | Mouse |
| meis1 | TGACA | Human |
| meis1 | (C/T/A)T(C/A)AATCA | Human |
| neurod2 | CATATG | Human |
| nkx2-1 | A_CCACTT(G/A/C)A(A/C)_TT (SEQ ID NO: 20) | Mouse |
| Olig1 | (A/G)(A/C)CATATG(T/G/C)(T/C) | Human |
| Olig2 | (A/G)(C/A)CATATG(G/T/C)(T/C) | Human |
| osr1 | GCTAC(T/C)GT | Human |
| otx1 | TAATCC | Human |
| otx2 | TAATCC | Human |
| otx2 | GGATTA | Human |
| pax2 | GTCACG | Mouse |
| pax6 | TT(C/T)ACGC(A/T)T(G/C)A_TT (SEQ ID NO: 21) | Human |
| POU3F2 | TATG(C/T)(A/T)AAT | Human |
| POU3F3 | TATG(C/T)(T/A)AATTT | Human |
| Ptf1a | (A/C/G)CAGATGTT | Mouse |
| Ptf1a | (A/C/G)CAGCTGT | Mouse |
| Ptf1a | (A/G/C)CACCTGT | Mouse |
| runx1 | TG(T/C)GGTT | Human |
| runx1 | TGTGGTT | Mouse |
| Runx1 | TGTGGTT | Human |
| Runx1 | (C/T)TGTGGTTT | Mouse |
| Runx3 | (A/T/C)(A/G)ACC(G/A)CAAA | Human |
| Runx3 | ACC(T/A) | Human |
| rxrg | (G/A)GGTCAAAGGTCA (SEQ ID NO: 22) | Human |
| rxrg | GGTCA___(A/G)GGTCA (SEQ ID NO: 23) | Human |
| rxrg | (A/G)GGTCA(T/C)GACC(T/C)(C/T) | Human |
| shox2 | (T/C)AATTA | Mouse |
| six3 | GGGTATCA | Mouse |
| smad4 | GTCT(A/G)G | Mouse |
| Sox1 | acaat(A/T/G)_cattgtt (SEQ ID NO: 24) | mouse |
| sox5 | ATTGTT | Mouse |
| sox8 | AACAAT(G/A)T(G/T)CAGTGTT (SEQ ID NO: 25) | Human |
| sox9 | ATTGTT | Human |
| sp8 | (C/A)(C/A/T)(A/C)C(G/T/C)CCC(A/C)(C/T)(T/C/A) | Human |
| SP9 | c(a/c)cgccc(c/a)(c/t) | Human |
| stat1 | GGAAA__GAAACT (SEQ ID NO: 26) | Human |
| stat1 | TTCC_GGAA | Human |
| stat1 | TTC(C/T)(A/G)GGAA | Human |
| stat1 | (A/G)(G/C)TTTC__TT(T/C)(C/T)(C/T) | Human |
| stat3 | TTCC(A/G/T)GGAA(G/A) | Mouse |
| stat3 | TTCTGGGAA | Human |
| tbr1 | AGGTGTGAA | Human |
| tbx5 | AGGTGTGA | Human |
| TLX1 (NFIC) | TGGCA___GCCAA (SEQ ID NO: 27) | Human |
| TLX3 | AATTG_______CAATT (SEQ ID NO: 28) | Human |
| vax2 | (C/T)TAATTA | Human |
| vax2 | (C/T)TAATTA | Human |
| vax2 | TAATTA | Mouse |
| ZBTB20 | (A/g)A(T/C)GTAT(A/G)(G/T/C) | Human |
| ZBTB7C | CGACCACCGA (SEQ ID NO: 29) | Human |
| Zfp105 | (C/T)AA(A/T)(C/T/A)AA(C/A)AA | Mouse |
| Zfp105 | (T/C)(G/T)_(T/G/C)TCAATAA_T(T/C)T | Mouse |
| Zic2 | CAGCAGG | Mouse |
| Zic2 | CACAGCAGG | Mouse |
| Zic2 | C_CAGCAGG | Mouse |
| Zic2_1 | CCCCCGGGGGGG (SEQ ID NO: 30) | Mouse |
| zic3 | (A/G/C)CTCCCCCCGC(T/G)G(C/T)G | Human |
| | (SEQ ID NO: 31) | |
| zic3 | (C/A)CCCCCGGGGGGG (SEQ ID NO: 32) | Mouse |
| zic3 | C(A/G)CAGCA(G/T)G | Mouse |

### Example 2

### Use for Spinal Muscular Atrophy (SMA)

Spinal muscular atrophy is a severe neurological disorder that results from loss of function mutations occurring in the SMN1 (SMN telomeric) gene which encodes the SMN protein. In humans, there is a secondary gene capable of encoding the SMN protein, the SMN2 (SMN centromeric) gene. The SMN2 gene is identical to the SMN1 gene except that it features a C to T substitution at position 840 that typically generates a variant of SMN lacking exon 7. However, approximately 10-15% of transcripts produce a full length SMN protein. SMA disease severity is dependent on the number of copies of SMN2 that patients have.

Multiple therapies for SMA have been developed over the past decade including Spinraza [Finkel et al., N. Engl. J. Med., 377.1723-1732 (2017), and Zolgensma [Mendell, et al., N. Engl. J. Med., 377: 1713-1722 (2017)]. Using these as positive controls for initial proof of concept experiments, the efficacy of six RPMs is demonstrated.

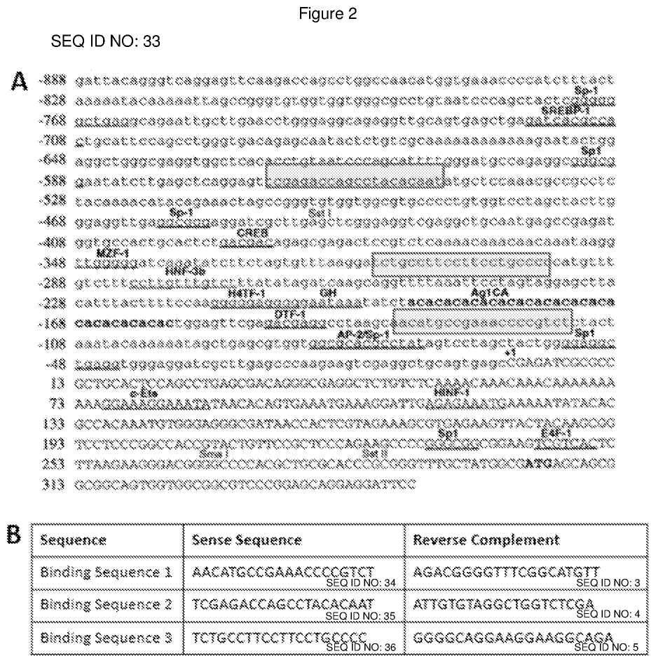

The six RPMs included one of three binding sequences to the SMN2 promoter. The promoter is described in Monani & Burghes, Biochim. Biophys. Acta—Gene Struct. Expr., 1445: 330-336 (1999). The three binding sequences targeted regulatory/binding sites outside of previously identified regulatory elements/binding sites for transcription factors (FIG. 2).

The six RPMs also included one of two endogenous regulatory elements (forward approach). One was a spinal cord small endogenous regulatory element, chr19:1479140-1479160 termed regulatory element A (CAGTCGCCGGCCTCTTCCCA) (SEQ ID NO: 1 reverse orientation). The other was a larger, pan-neuronal regulatory element, chr5:176943840-176943910 termed regulatory element B (TCGAGACCAGCCTACACAATAAGCACACGCCAGTCAGGCACGGCCTGCTCCCCTCTT CACGCCCGCTCCCACACGCGCGGGGTCTCGCCT) (SEQ ID NO: 2 reverse orientation). Both regulatory elements attract transcription factors to increase SMN2 expression.

The sequence encoding the combined regulatory element tail and binding sequence of each of the six RPMs A1, A2, A3, B1, B2 and B3 is shown at the right in FIG. 3. The sequences are in the reverse orientation so that the binding sequence targets the sense strand of the SMN2 promoter.

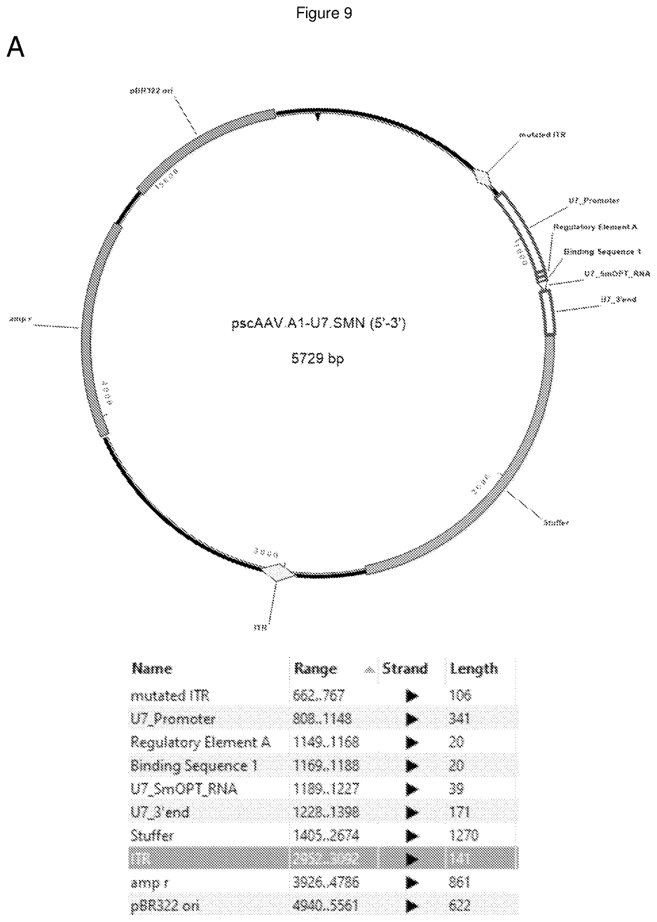

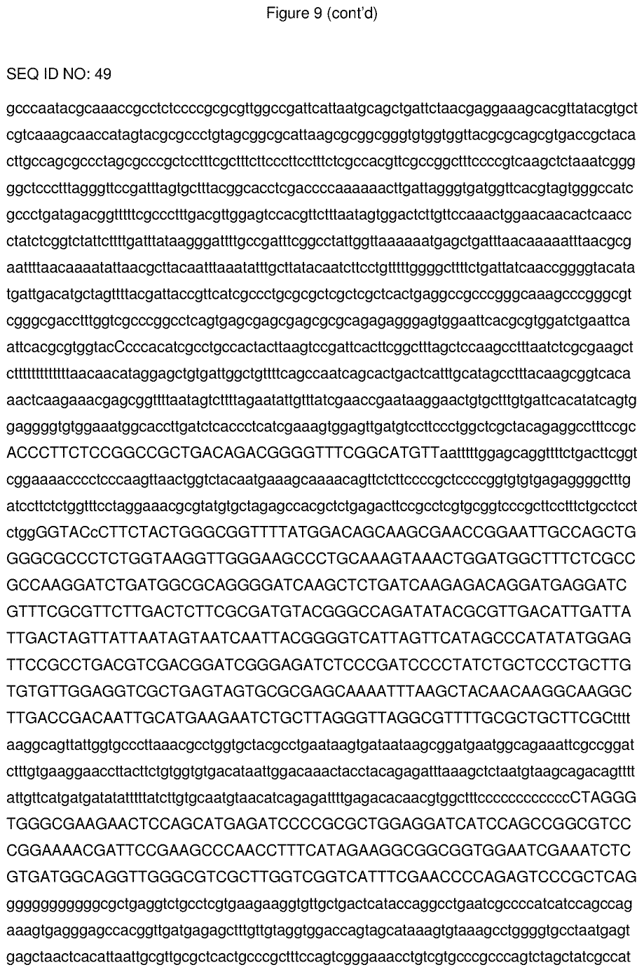

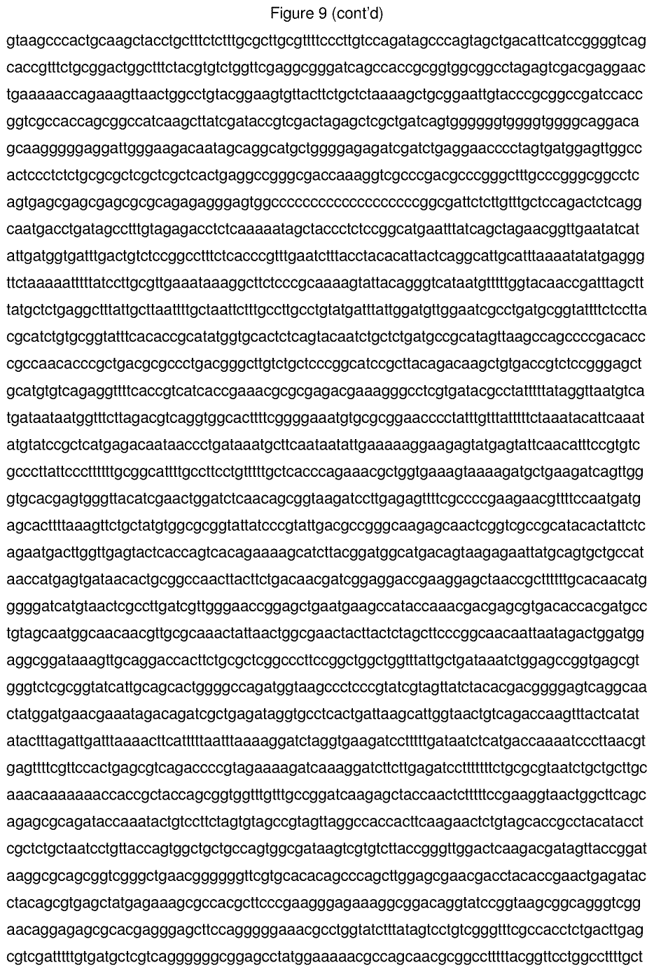

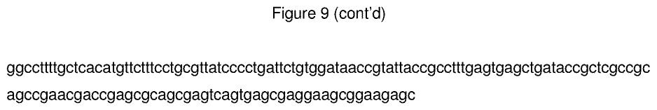

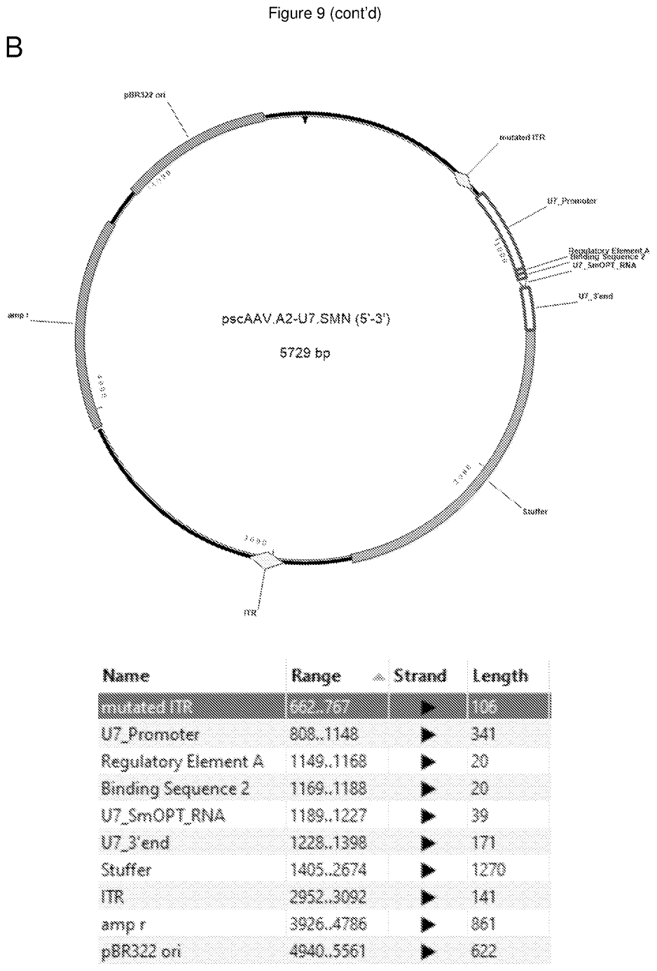

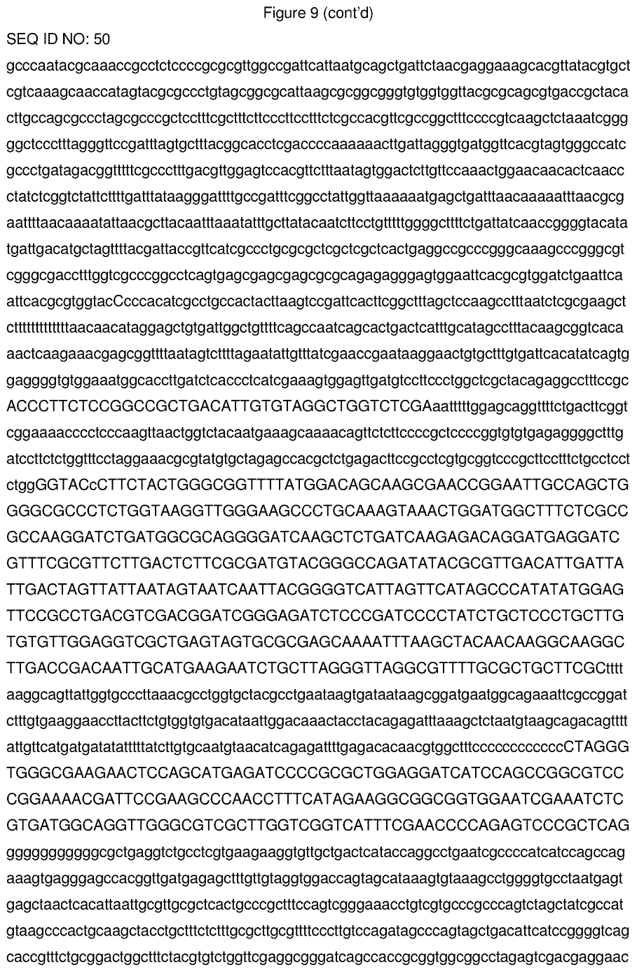

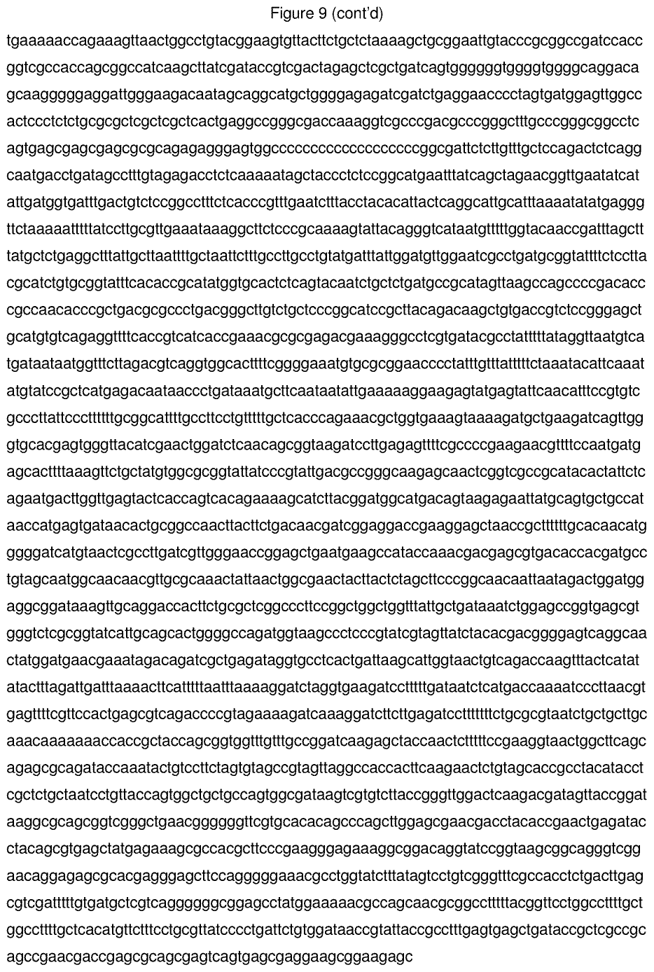

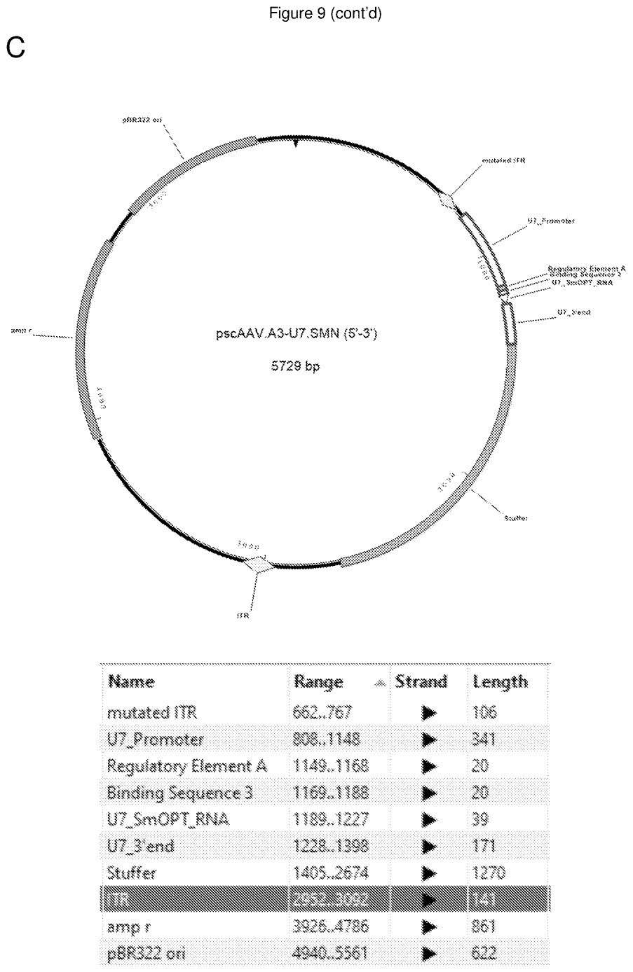

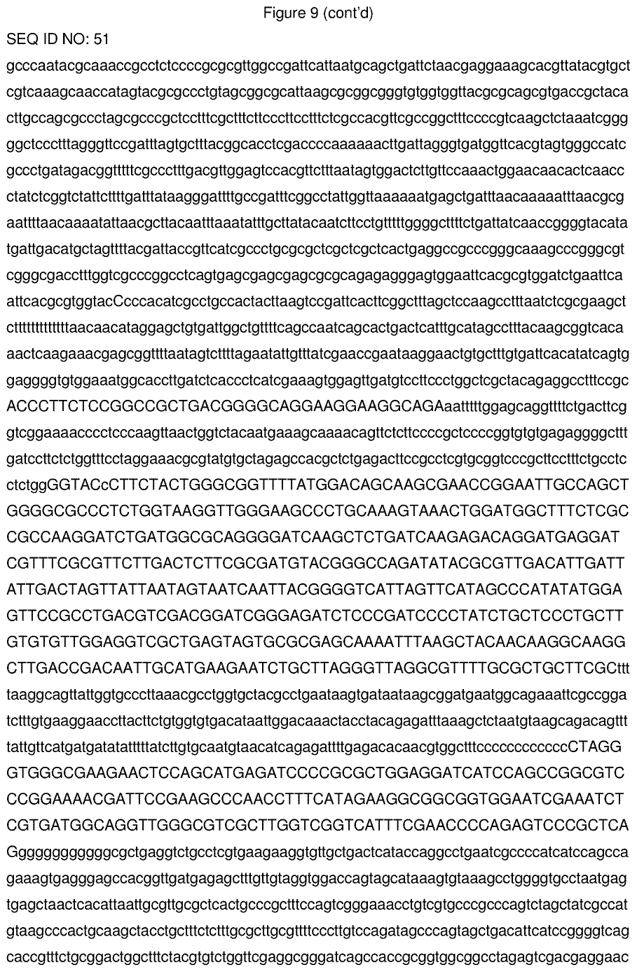

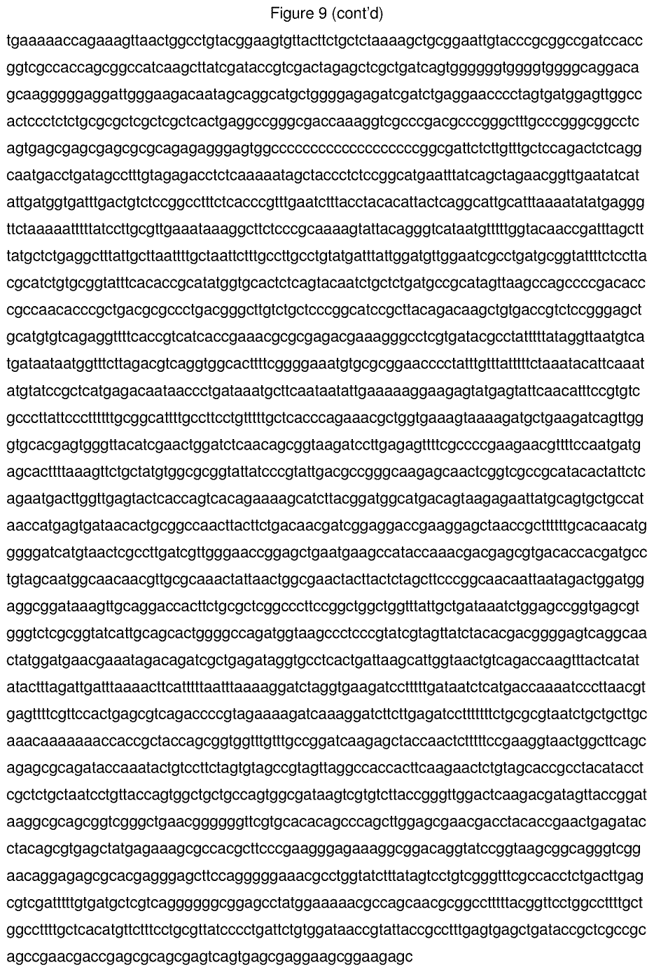

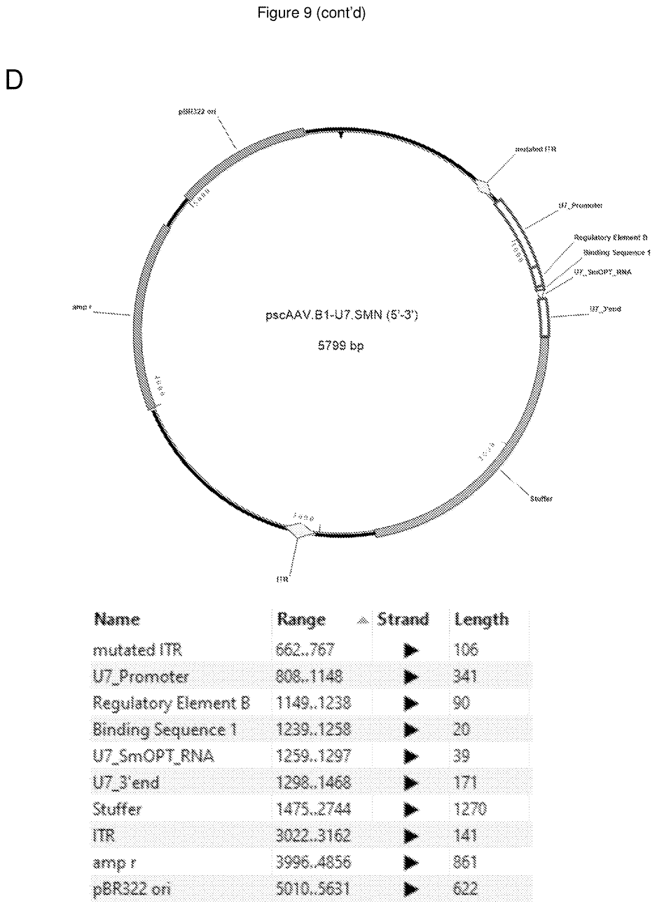

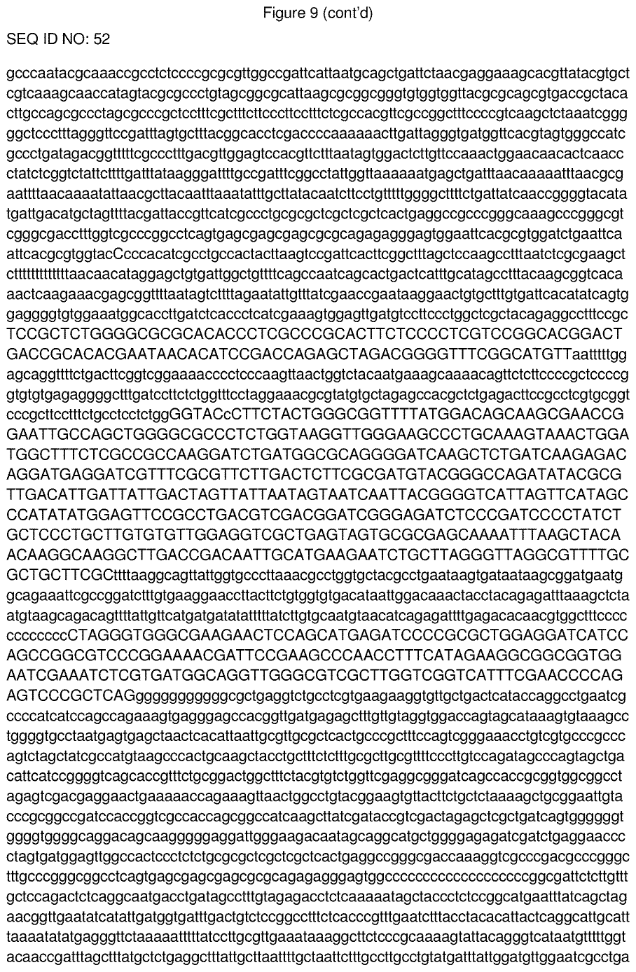

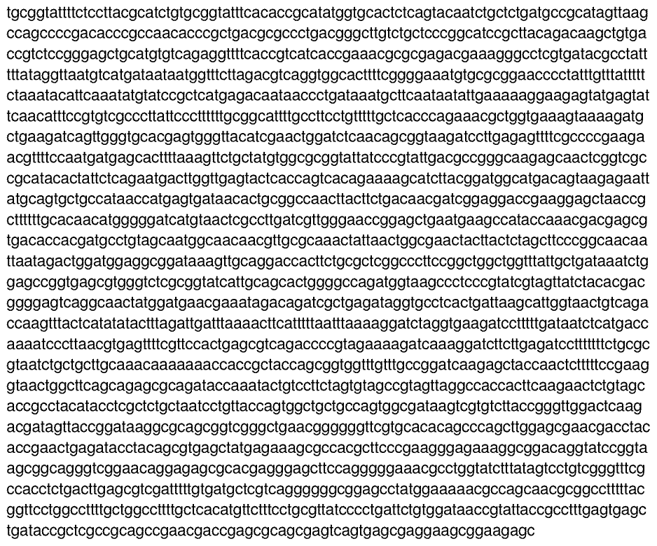

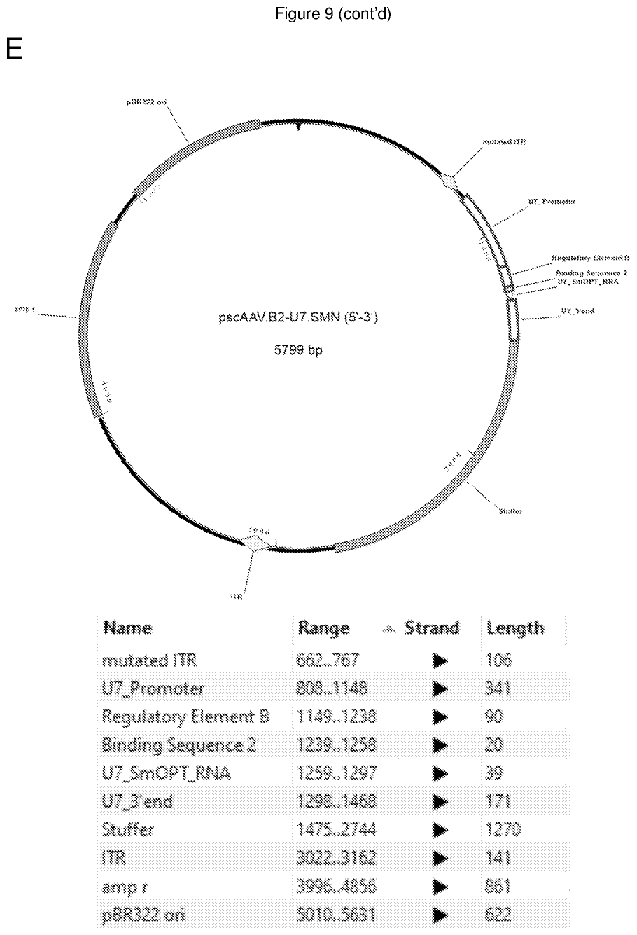

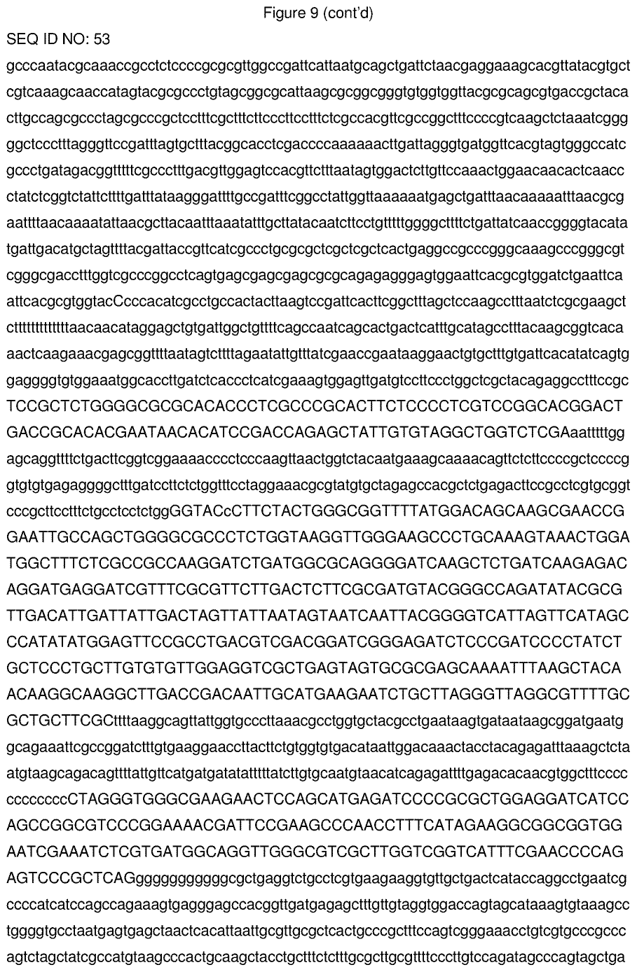

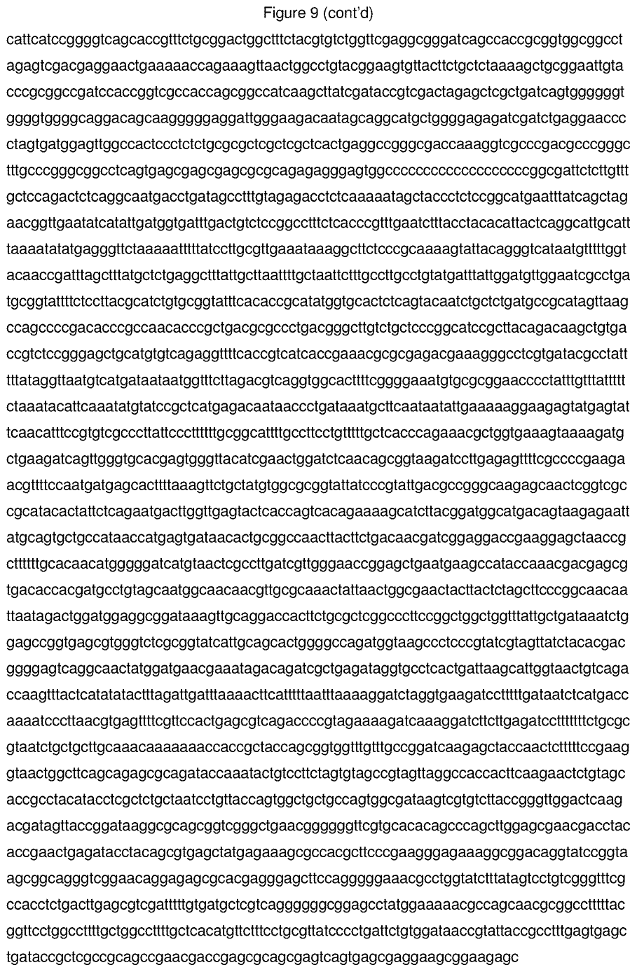

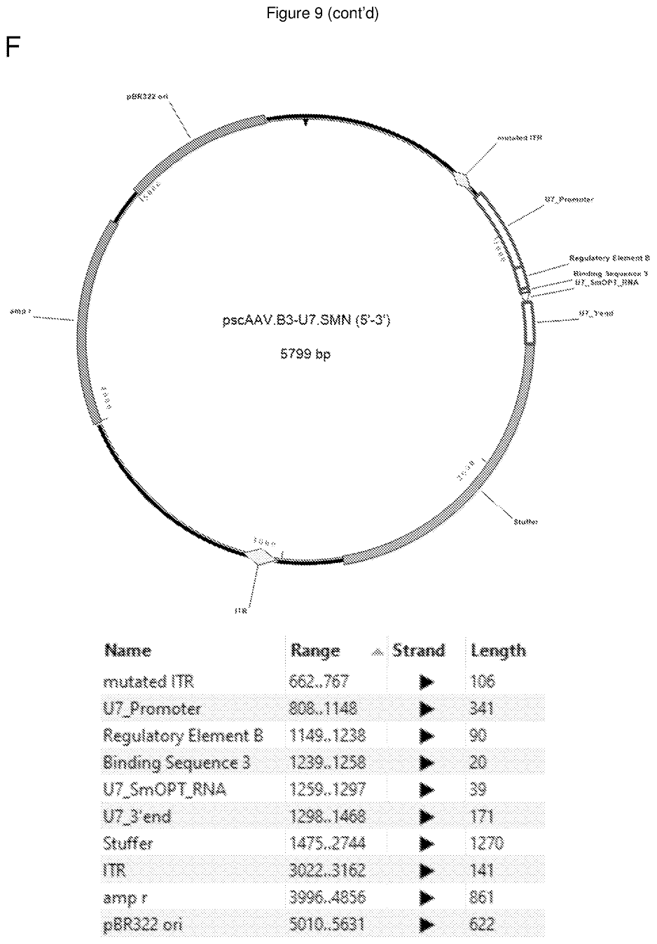

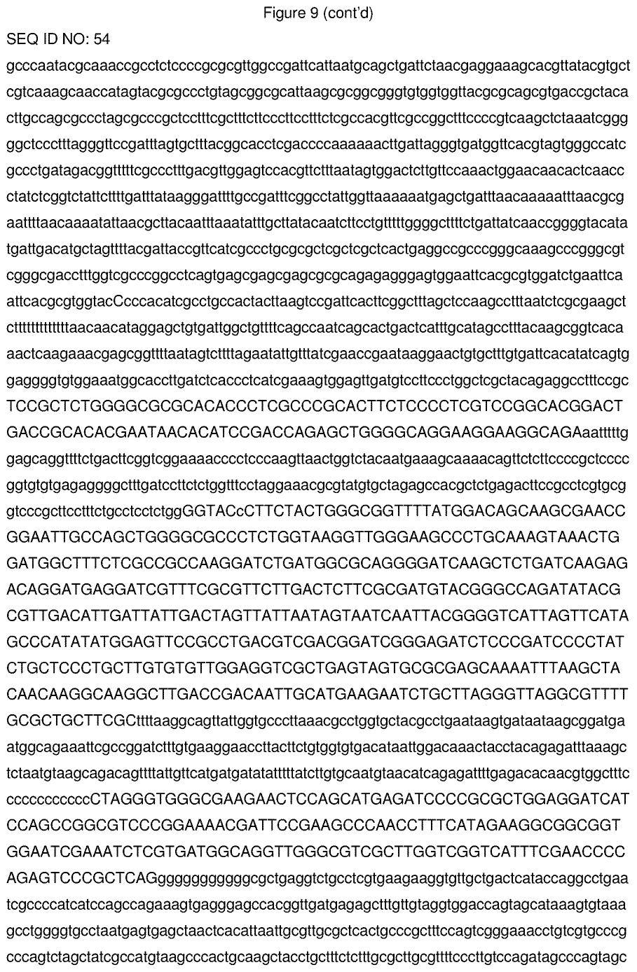

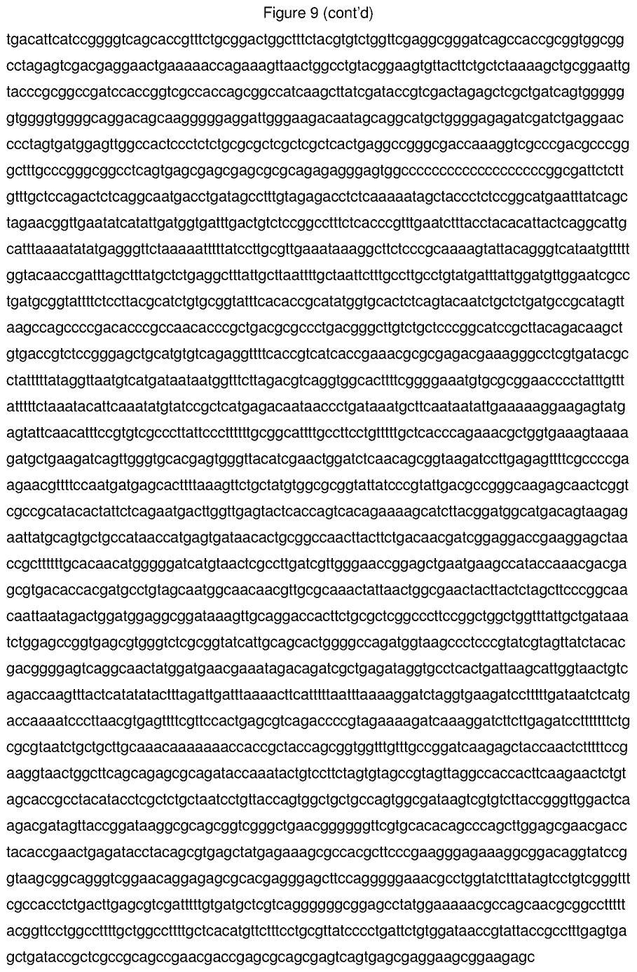

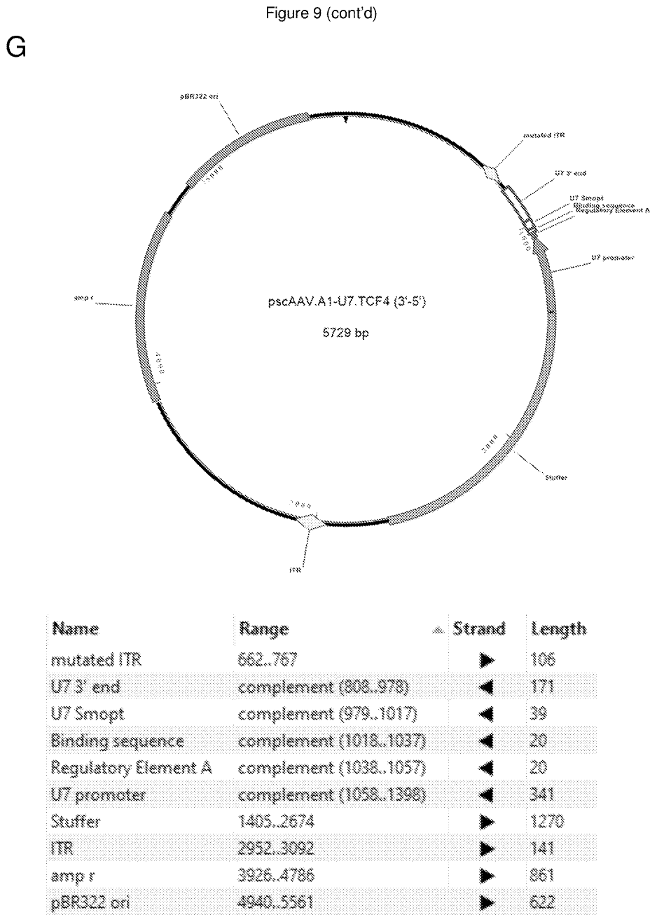

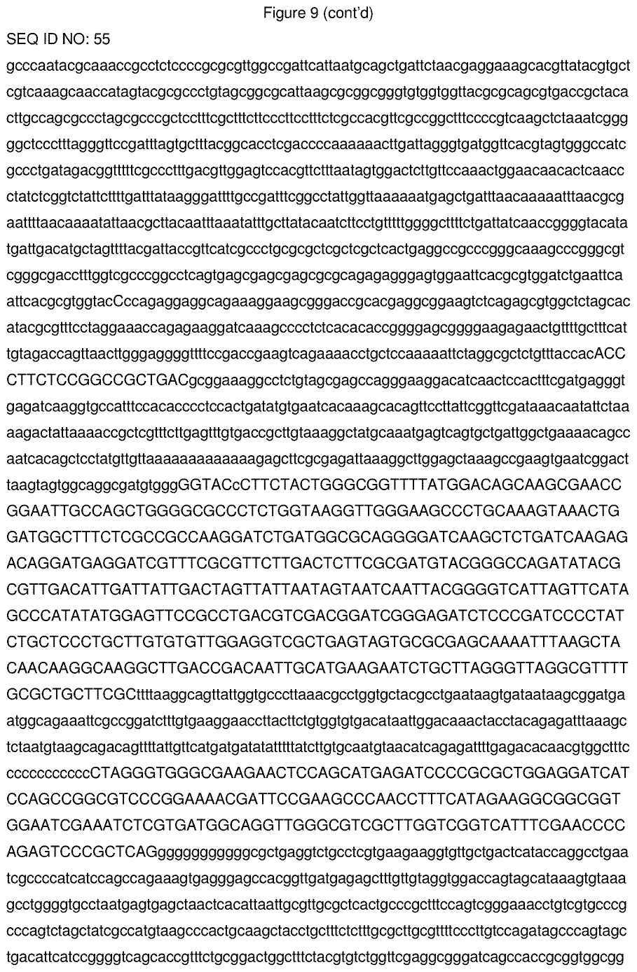

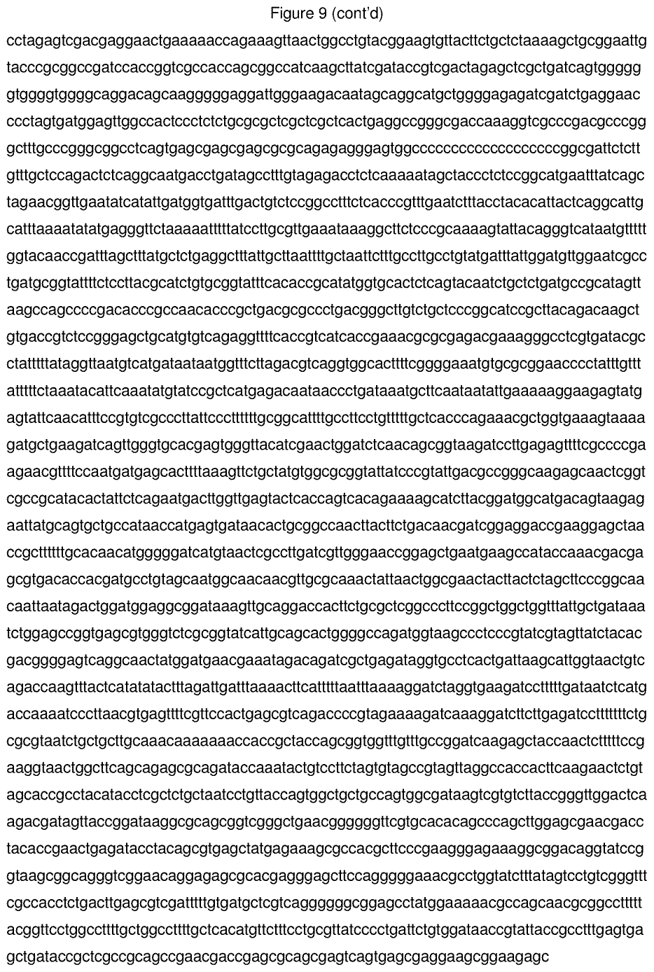

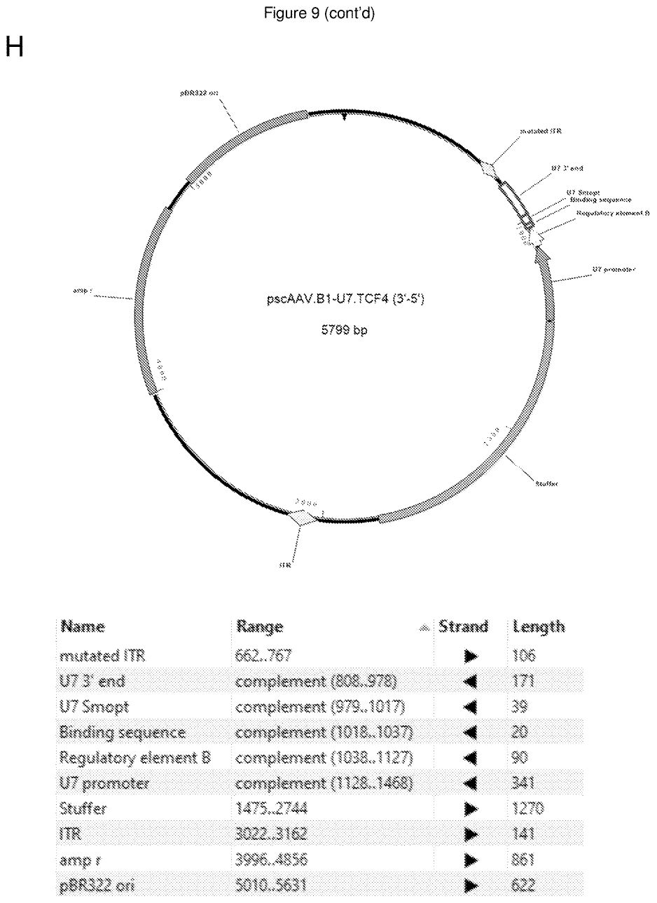

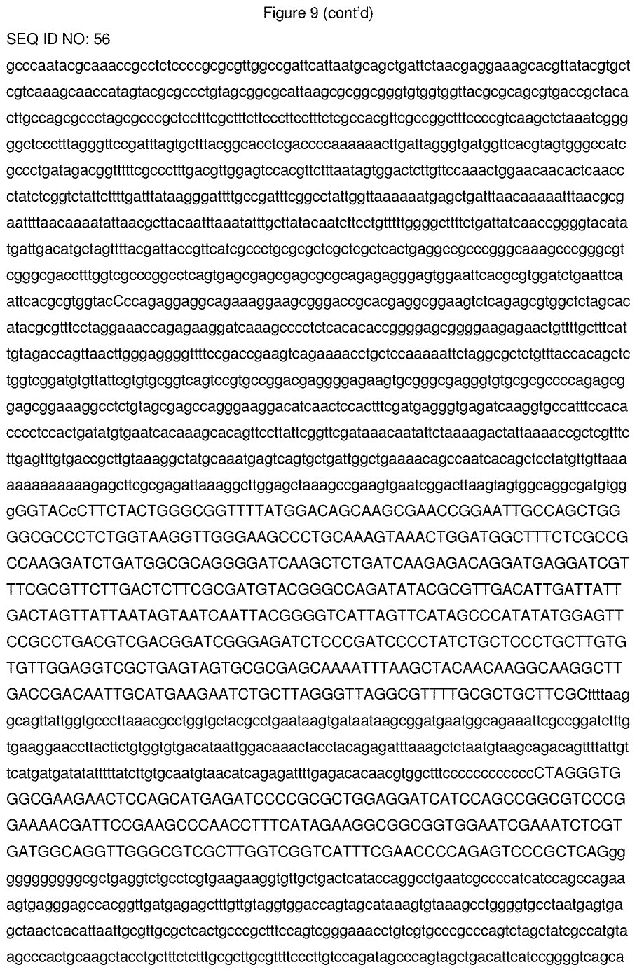

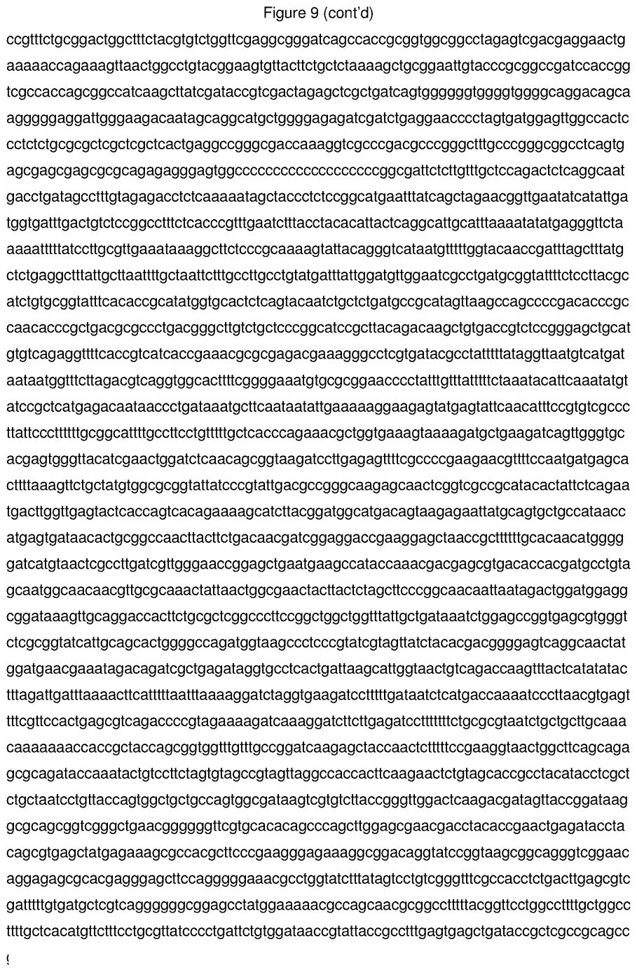

The RPMs are expressed from rAAV. Production plasmids for the six RPMs are respectively shown in FIG. 9A-F.

The rAAV are used to transfect humanized SMA model mouse embryonic fibroblasts (MEFs) and human SMA patient fibroblasts. SMN2 transcript expression in the cells is then measured by qPCR and/or ddPCR.

### Example 3

### Use for Pitt Hopkins Syndrome

Pitt Hopkins Syndrome (PTHS) is a rare neurodevelopmental disorder characterized by developmental delays, distinctive facial features, apneic episodes, and a broad spectrum of behavioral symptoms. Haploinsufficiency caused by mutations in the TCF4 gene resulting from either a pathogenic variant in TCF4 or a deletion of the chromosome region in which TCF4 is located (18q21.2). To date, there is no treatment or cure available for PTHS. The TCF4 gene is a large gene that encodes for a transcription factor with multiple isoforms, thus making it a challenging target for classical gene replacement therapy approaches. RPM therapy acts on the endogenous promoter to enhance the transcription, so PTHS is an ideal indication for RPM therapy due to the haploinsufficient nature of the disease. An RPM construct is provided herein to bind the single available TCF4 copy and enhance the transcription of TCF4 to normal levels.

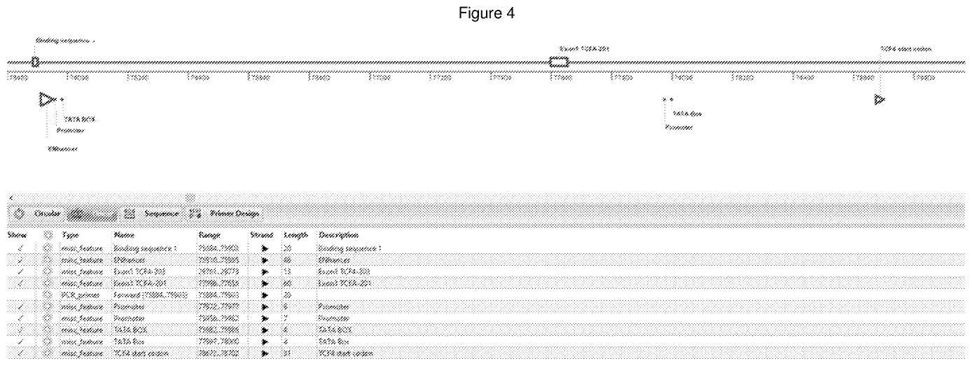

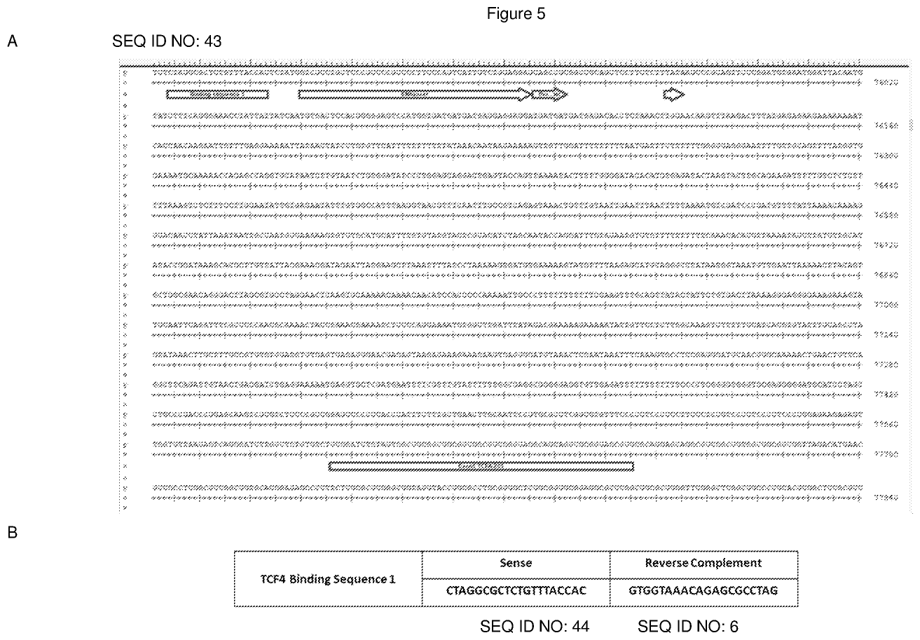

Preliminary binding sequences to the TCF4 promoter were designed. The TCF4 promoter had not been previously characterized, but utilizing bioinformatics tools for promoter analysis, the putative TATA box and enhancer elements were identified within the 2000 bp region upstream of Exon 1 of the TCF4-201 transcript variant (FIG. 4). The 20 bp sequence of these putative elements was chosen as the binding site. (FIG. 5).

Two TCF4 RPM constructs were generated utilizing two different regulatory elements and targeting the identified 20 bp binding site (FIG. 6). Initial testing was done using endogenous regulatory elements (forward approach), specifically a spinal cord small endogenous regulatory element which would not hinder U7 small nuclear RNA function due to size, and a larger, pan-neuronal regulatory element which is present throughout different target tissues in the central nervous system. The RNAfold webserver can be used to calculate RNA secondary structures based on minimum free energy calculations and centroid structure calculations [Gruber et al., The Vienna RNA Websuite, Nucleic Acids Res., 36 (Web Server issue): W70-74 (2008)] for selecting which regulatory elements to use based on the predicted secondary structures. The spinal cord element chosen was chr19:1479140-1479160, referred to as regulatory element A herein, and the pan-neuronal element chosen was chr5:176943840-176943910, referred to as regulatory element B herein. For each of these, a construct was designed with the binding sequence targeting the TCF4 gene (FIG. 6).

As explained in the Summary section above, the constructs were designed so that the binding site of the RPMs is capable of binding the 5′ DNA strand. The regulatory tail was oriented so that the proteins binding the regulatory element will also be in contact with the 5′ DNA strand of the promoter. Furthermore, the entire U7 construct was assembled so that the sm OPT and hairpin are downstream of the promoter so that these U7 construct components do not interfere with transcription factor or RNA polymerase II binding to the gene and transcription. Therefore, the RPMs are designed so that the binding sequence is in the reverse complement orientation of the DNA 5′ binding site and the regulatory element tail is in the reverse orientation of the endogenous regulatory element (e.g., transcription factor binding site). The DNA sequences encoding the two constructs (A1.U7.TCF4 and B1.U7.TCF4) targeting the TCF4 gene are shown in (FIG. 7).

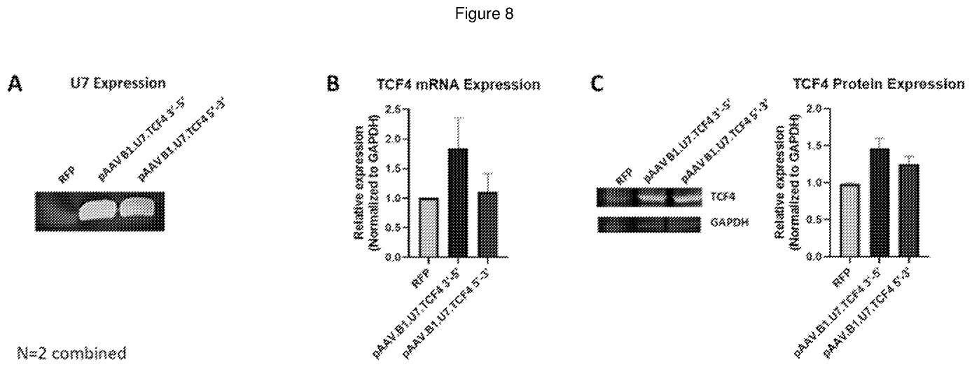

Plasmids containing constructs A1.U7.TCF4 and B1.U7.TCF4 were ordered from Genscript. Each construct contains U7 promoter-Regulatory element tail coding sequence-binding sequence coding sequence-smOPT/hairpin sequence-U7 Terminator sequence (shown respectively in FIG. 7). The entire U7 expression cassette was further cloned in pscAAV.stuffer plasmid in both orientations (3′-5′ and 5′-3′). The plasmids (FIG. 9G,H) were tested for U7 expression as well as TCF4 expression (both mRNA and protein) in HEK293 cells 72 hrs post transfection (FIG. 8A, B, C, respectively).

The plasmids were further used for production of scAAV9.U7.TCF4 vector. Self-complementary AAV were produced by transient transfection procedures using each plasmid, along with a plasmid encoding Rep2Cap9 sequence along with an adenoviral helper plasmid pHelper (Stratagene, Santa Clara, CA) in 293 cells. The vectors will be used to transduce mouse embryonic fibroblasts (MEFs) carrying humanized TCF4 promoter driving eGFP+LUciferase+TCF4 and human TCF4 patient-derived cells. U7 transcript expression as well as TCF4 mRNA expression by qPCR and/or ddPCR will demonstrate the vectors are functional and able to increase TCF4 transcript production. Staining and western blot analysis will demonstrate increased TCF4 protein expression. For clinical translation of the AAV9.U7.TCF4 vector, rescue experiments in TCF4 patient-derived cells will be performed to demonstrate the therapeutic potential of the treatment on astrocyte differentiation, seahorse analysis and motor neuron cocultures.

The data from HEK 293 cells shows that the B1.U7.TCF4 construct is successful in increasing the TCF4 mRNA and protein levels and it is contemplated herein that the same construct will provide therapeutic benefits.

### Example 4

### Additional Experiments—Use for Pitt Hopkins Syndrome

As mentioned in Example 3, hTcf4-H2B-EGFP-Nluc MEF cell lines were established from transgenic mice. The transgenic mice comprised a humanized TCF4 promoter driving concurrent expression of mouse TCF4, GFP and luciferase. HOMO MEFs represent the mouse embryonic fibroblasts homozygous for the hTCF4 promoter-driven expression. HOMO hTCF4 MEFs were cultured in DMEM+10% FBS+1% Anti/Anti medium and transduced with scAAV9.B1.U7.TCF4 with insulin treatment. scAAV9.RFP vector was used as transduction control. 72 hours after transduction, the cells were collected to harvest RNA, followed by cDNA preparation. qPCR was performed to determine the B1.U7 expression as well as msTCF4 expression.

qPCR data revealed that the treatment of humanized TCF4 MEFs with scAAV9.B1.U7.TCF4 resulted in increased expression of U7.B1 along with concurrent increase in total TCF4 expression levels as compared to the cells treated with scAAV9.RFP thus, again demonstrating RPM effects on transcription. See FIG. 9.

To further demonstrate the use of U7 promoter modulation in the context of PTHS, an in vitro disease model of induced astrocytes from PTHS patients was established. Skin biopsies from three PTHS patients carrying different mutations in the TCF4 gene were obtained to establish skin fibroblasts cultures. These fibroblasts were further converted to induced neuronal progenitor cells (iNPCs) using a direct conversion protocol. TCF4 iNPCs were further differentiated into induced astrocytes (iAs) using published protocols. See, for example, Meyer et al., Proc. Natl. Acad. Sci. USA, 111(2):829-832 (2014). scAAV9.B1.U7.TCF4-treated induced astrocytes were subjected to TCF4 expression analysis, 5 days post transduction. iAs lines derived from two healthy individuals were treated as controls.

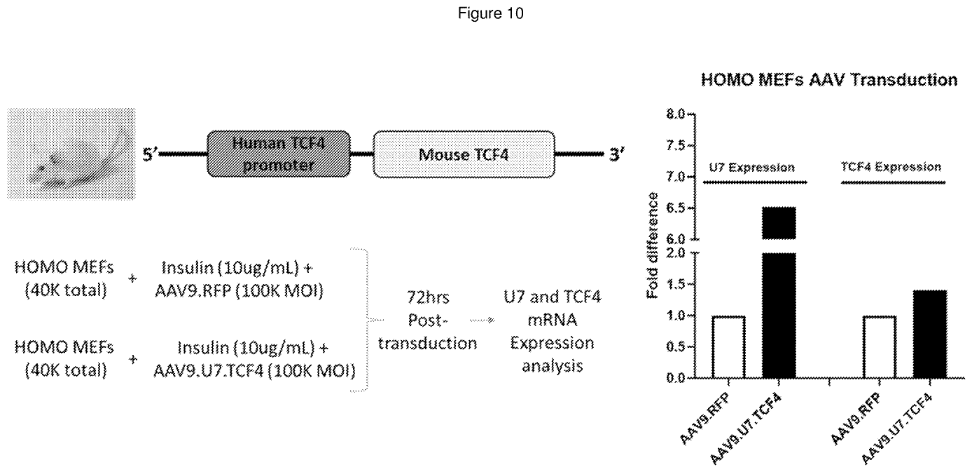

TCF4 mRNA expression in scAAV9.B1.U7.TCF4 treated cells was analyzed using qPCR analysis. FIG. 10 shows TCF4 patient-derived iAs show reduced mRNA expression of TCF4 as compared to healthy control iAs. Treatment with scAAV9.B1.U7.TCF4 resulted in a significant increase in TCF4 expression in one patient line (3944) with modest but statistically insignificant increase in TCF4 mRNA expression in other lines (6091, 6038).

TCF4 protein expression was also examined in all three lines using immunofluorescence analysis. scAAV9.B1.U7.TCF4-treated patient and control iAs were stained with primary TCF4 Antibody (ab185736, Abcam) at 1/100 dilution, followed by appropriate secondary antibody. The cells were imaged using Nikon Eclipse Ti2-E fluorescent microscope at 40× magnification. Images from 15 random fields were subjected to measurement of area and fluorescent intensity using Fiji. TCF4 intensity per cell was calculated and compared between the treated and untreated cell lines with Graphpad. The entire experiment was repeated 6 times.

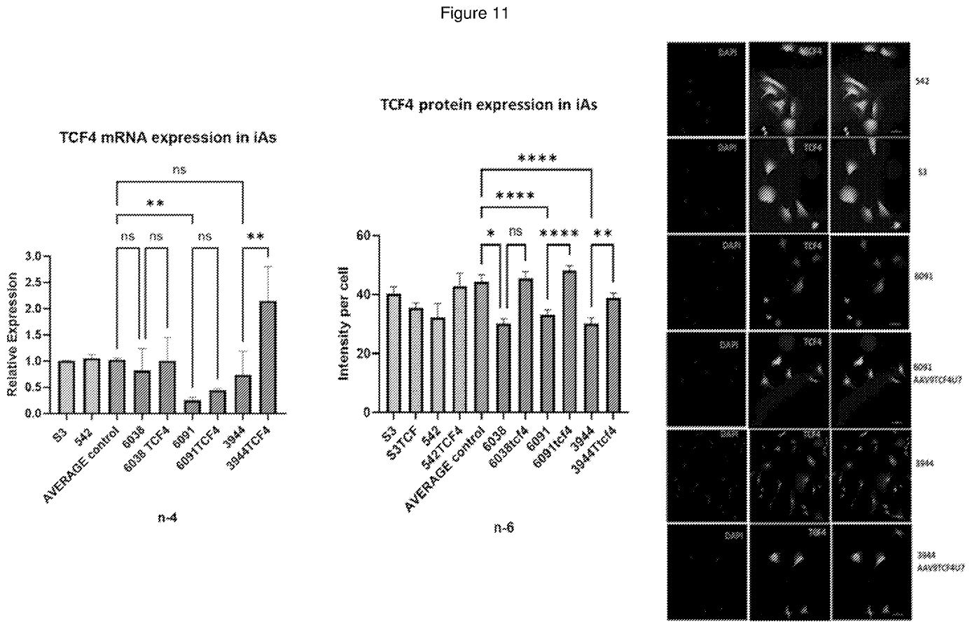

Results are shown in FIG. 11. Similar to mRNA expression, TCF4 iAs show reduced TCF4 protein expression as compared to healthy iAs. scAAV9.B1.U7.TCF4 treatment resulted in rescue of TCF4 expression in all three patient lines (6038, 6091 and 3944) with increased TCF4 signal in these lines as compared to their untreated counterparts.

Finally, efficacy of scAAV9.B1.U7.TCF4 was tested in another in vitro disease model for PTHS. This in vitro disease model of PTHS consists of coculture of PTHS patient-derived induced astrocytes with mouse HB9-GFP neurons. The coculture system allows determination of the patient iAs-mediated toxicity towards the neurons as compared with healthy iAs. Coculture experiments were performed with PTHS-induced iAs treated or untreated with scAAV9.B1.U7.TCF4. Two iAs lines from two healthy individuals were used as controls. The experiment was repeated 6 times.

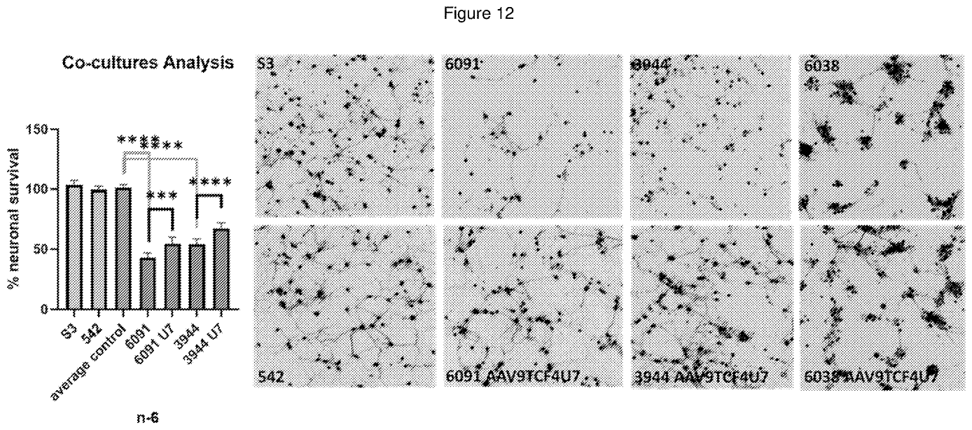

The coculture data (FIG. 12) demonstrated that two of the PTHS iAs (6091, 3944) are significantly toxic to GFP neurons as compared to healthy iAs (S3 and 542). Importantly, treatment of patient iAs with scAAV9.B1.U7.TCF4 resulted in significant rescue of neuron survival.

Collectively, the TCF4 expression and coculture data demonstrates that scAAV9.B1.U7.TCF4 treatment of PTHS iAs results in elevated TCF4 expression that leads to rescue of neuronal survival in PTHS iAs. Thus, the RPMs modulate transcription of target genes as contemplated.

We claim:

1. An RNA-based promoter modulator (RPM) comprising:
(a) a regulatory element tail,
(b) a binding sequence, and
(c) an Sm OPT consensus sequence and hairpin.

2. The RPM of claim 1 wherein the orientation of (a), (b) and (c) in the RPM is 5′ to ‘3’ and wherein the (b) binding sequence binds the sense strand of a target gene promoter.

3. An SMN2 RPM comprising:
(a) Regulatory Element A Tail encoded by
(SEQ ID NO: 1)
ACCCTTCTCCGGCCGCTGAC
or
Regulatory Element B Tail encoded by
(SEQ ID NO: 2)
TCCGCTCTGGGGCGCGCACACCCTCGCCCGCACTTCTCCCCTCGTCCGG
CACGGACTGACCGCACACGAATAACACATCCGACCAGAGCT,
and
(b) Binding Sequence 1 encoded by
(SEQ ID NO: 3)
AGACGGGGTTTCGGCATGTT,
Binding Sequence 2 encoded by
(SEQ ID NO: 4)
ATTGTGTAGGCTGGTCTGA,
or
Binding Sequence 3 encoded by
(SEQ ID NO: 5)
GGGGCAGGAAGGAAGGCAGA.

4. A TCF4 RPM comprising:
(a) Regulatory Element A Tail encoded by
(SEQ ID NO: 1)
ACCCTTCTCCGGCCGCTGAC
or
Regulatory Element B Tail encoded by
(SEQ ID NO: 2)
TCCGCTCTGGGGCGCGCACACCCTCGCCCGCACTTCTCCCCTCGTCCGG
CACGGACTGACCGCACACGAATAACACATCCGACCAGAGCT,
and
(b) Binding Sequence 1 encoded by
(SEQ ID NO: 6)
GTGGTAAACAGAGCGCCTAG.

5. A nucleic acid encoding an RPM, the nucleic acid comprising
(a) a DNA encoding
(i) an RPM of claim 1 or 2,
(ii) an SMN2 RPM of claim 3, or
(iii) a TCF4 RPM of claim 4;
(b) a DNA construct of FIG. 7A or 7B; or
(c) a DNA at least 90% identical to the DNA of (a) or DNA construct of (b).

6. The nucleic acid of claim 5 (a) further comprising a promoter driving expression of the RPM.

7. The nucleic acid of claim 6, wherein the promoter is any of a U7 promoter, U6 promoter, a tRNA promoter, a H1 promoter, a minimal CMV promoter, a T7 promoter, an EF1-alpha promoter, a Minimal EF1-alpha promoter.

8. A recombinant adeno-associated virus (rAAV) comprising the nucleic acid of any one of claims 5-7.

9. The rAAV of claim 8, wherein the AAV is a single-stranded AAV (ssAAV) or a self-complementary AAV (scAAV).

10. The rAAV of any one of claims 8-9, wherein the rAAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh.10, AAV-B1, MyoAAV, MYOAAV1A, SLB101, M1 and M3, or their derivatives.

11. The rAAV of any one of claims 8-10, wherein the rAAV is rAAV9.

12. A composition comprising
(a) the nucleic acid of any one of claims 5-7, or
(b) the rAAV of any one of claims 8-11, and
a pharmaceutically acceptable carrier.

13. A method of modulating expression of a target gene in a cell comprising delivering an RPM of any of claims 1-4 to the cell.

14. A method of modulating expression of a target gene in a cell comprising contacting the cell with:
(a) the nucleic acid of any one of claims 5-7,
(b) the rAAV of any one of claims 8-11, or
(c) the composition of claim 12.

15. The method of claim 14 wherein the expression of the target gene increases.

16. The
(a) nucleic acid of any one of claims 5-7,
(b) adeno-associated virus (AAV) of any one of claims 8-11, or
(c) composition of claim 12,
wherein the nucleic acid, AAV or composition is formulated for intramuscular injection, intrathecal injection, transdermal transport or injection into the blood stream.

17. A method of treating spinal muscular atrophy in a subject comprising delivering a SMN2 RPM of claim 3 to the subject.

18. A method of treating Pitt Hopkins Syndrome in a subject comprising delivering a TCF4 RPM of claim 4 to the subject.
